(19) United States
(12) Patent Application Publication
(10) Pub. No.: US 2011/0165601 A1
(43) Pub. Date: Jul. 7, 2011

(54) ASSAY SYSTEM

(75) Inventors: Godfrey L. Smith, Glasgow (GB); Jon Cooper, Greater London (GB)

(73) Assignee: THE UNIVERSITY COURT OF THE UNIVERSITY GLASGOW

(21) Appl. No.: 12/742,216

(22) PCT Filed: Nov. 9, 2007
(86) PCT No.: PCT/GB07/04307

§ 371 Date: Mar. 22, 2011

(30) Foreign Application Priority Data
Nov. 10, 2006 (GB) 0622475.2

(51) Int. Cl. C12Q 1/48; C12N 5/077; C12Q 1/02; C12Q 1/34

(52) U.S. Cl. 435/15; 435/374; 435/29; 435/18

(57) ABSTRACT

The present invention relates to materials and methods for performing assays directed to monitoring activity and function of intracellular components, such as proteins associated with organelles and other intracellular structures. In particular, the invention relates to permeabilised cell preparations and their use in studying activity of intracellular components, in particular for studying sarcoplasmic reticulum function.

### FIELD OF THE INVENTION

The present invention relates to materials and methods for performing assays directed to monitoring activity and function of intracellular components, such as proteins associated with organelles and other intracellular structures. In particular, the invention relates to permeabilised cell preparations which retain intracellular activity over long periods of storage, and their use in studying activity of intracellular components. In preferred embodiments the invention relates to materials and methods for studying sarcoplasmic reticulum function, which are particularly useful in research directed to designing drugs for heart disease and studying potential cardiac side-effects of drugs for other conditions.

### BACKGROUND OF THE INVENTION

Heart Failure is a common clinical problem with high morbidity and mortality. Left ventricular dysfunction (LVD) is present in around 3% of all adults, and is symptomatic in around half of this group (McDonagh et al, 1997). Gwathmey et al (1987) were the first to suggest that abnormal Ca²⁺ homeostasis in ventricular myocytes contributed to mechanical dysfunction in heart failure.

Subsequently, many studies in both human and animal models of heart failure have found reduced rate of decline of intracellular Ca²⁺ transients e.g. (Bing et al, 1991; Beuckelmann et al, 1992; Anversa et al, 1991). Pieske et al (Pieske et al, 1995) found reduced peak systolic Ca²⁺ levels which correlated with a negative force-frequency relationship in failing human trabeculae, and similar findings have been reported in other human and animal studies (Beuckelmann et al, 1992; Qin at al, 1996).

One focus of study has been the function of the sarcoplasmic reticulum (SR) in heart failure. Schwinger et al (Schwinger at al, 1995) found 50% lower Ca²⁺ uptake rates in isolated SR vesicles and reduced Sarco-endoplasmic reticulum Ca²⁺ ATPase (SERCA2) activity in human dilated cardiomyopathy. Similar findings have been reported in other human studies (Hasenfuss et al, 1994) and in animal models (e.g. (Afzal & Dhalla, 1992; Cory et al, 1993) using both tissue homogenates and vesicle preparations.

Protein abundance measurements for SR Ca²⁺ handling proteins are conflicting (see (Hasenfuss, 1998) and (Movsesian et al, 1989) for review). The majority of abundance measurements indicate a reduction in SERCA2 protein expression in parallel with Ca²⁺ uptake (Mercadier et al, 1990; Arai et al, 1993; Kiss et al, 1995; Matsui et al, 1995; Bartel et al, 1996; Currie & Smith, 1999). However, in human heart failure, reduced phosphorylation of phospholamban rather than reduced abundance may be a factor (Movsesian et al., 1989).

With regard to the SR Ca²⁺ release channel/ryanodine receptor (RyR2), some studies have shown no difference in protein expression (Movsesian et al., 1994) while others indicate reduced expression in heart failure (Meyer et al., 1995; Brillantes et al., 1992).

Measurements of RyR2 function using laser-scanning confocal microscopy have identified a reduced efficiency of coupling between Ca²⁺ influx and Ca²⁺ release from the SR in a rat model of cardiac hypertrophy but no obvious change in RyR2 function (Gomez et al., 1997). Yet recent work has shown that binding of the modulatory protein FKBP12.6 to RyR2 is reduced in human heart failure (Marx et al., 2000) and animal models (Ono et al., 2000). Reduced binding of FKBP12.6 to RyR2 affects the kinetics of the channel and therefore may alter excitation-contraction (E-C) coupling in failing hearts. However, total Ca²⁺ flux through the RyR2 in intact cardiac SR from failing hearts has not been examined directly. The remaining SR Ca²⁺ flux (non-RyR2 mediated Ca²⁺ leak) can potentially affect SR function.

The pre-eminence of heart disease in the western world has meant that the majority of large pharmaceutical companies have active research programs to develop novel drugs for the cardiovascular system. Recent work has highlighted a key defect in the failing heart as being predominately caused by the reduced effectiveness of the subcellular organelle, SR, in the heart muscle cell. This organelle is of more general interest to the pharmaceutical industry because the dysfunction of the SR is a known trigger of potentially lethal arrhythmias (Bers 2001). Recently legislation has been put in place that necessitates the screening of all drugs for their arrhythmogenic potential. However, despite being the focus of research, there is a paucity of experimental preparations to allow the investigation of SR function.

### SUMMARY OF THE INVENTION

The present inventors have appreciated that there is a need for an assay system that will facilitate research and drug design for heart disease. Given the strong evidence that sarcoplasmic reticulum (SR) function is an important factor when investigating heart failure, the inventors have concentrated on designing an assay system to follow SR activity, but the system is equally applicable to studying the activity and function of other intracellular organelles such as the nucleus and mitochondria, and structures such as the contractile proteins, and may also be applied to cell types other than myocytes. The system enables preparation of large batches of material which can be stored for long periods of time without significant loss of activity and which lend themselves particularly well to use in assays having a medium- or high-throughput format.

Existing assay systems for investigating SR function generally use membrane vesicle preparations that are not in their natural configuration and consequently have limited applicability to the intact cell. Typically a biochemical preparation of the SR is generated by homogenisation and centrifugation to produce a vesiculated form of the SR. The capacity of the SR to sequester Ca²⁺ is measured using a radioactive Ca²⁺ tracer or the generation of a metabolite that was the result of Ca²⁺ uptake (inorganic phosphate) (Coll et al 1999). A number of disadvantages are associated with this sort of preparation including: (a) the process of isolating the SR vesicles is time consuming and expensive; (b) using radioactive tracers is difficult and expensive; (c) the SR vesicle preparation does not remain viable, i.e. the activity of the preparation decreases progressively over the time course of the assay (approximately 30 minutes); and (d) the activity of the preparation is not sustained well even when stored at −80° C.

In one aspect, the present invention provides methods of preparing frozen stocks of cells, in particular permeabilised cells, in which the activity of organelles and other intracellular structures can be maintained during long periods of storage (up to 6 months, or even 9 months or longer) and which lend themselves well to use in medium- and high-throughput assay systems.

In medium- or high-throughput assays, it may be desirable to automate as many steps as possible while keeping the amount of manipulation required to a minimum. The cell preparations of the invention may be used easily and routinely in assays of 96-well format or higher. Typically, a medium-throughput assay will involve tests of 100 to 200 different compounds in a test system, with a relatively detailed data acquisition phase, e.g. establishing a time course for a particular effect. A high-throughput assay will typically involve 10,000 or more compounds, but may involve less detailed data acquisition, e.g. looking for the presence of absence of a desired effect.

Thus the invention provides a method for preparing a stock of cells for future use in an assay for determining activity or function of a target intracellular component, the method comprising contacting a population of cells with an effective amount of a permeabilising agent and freezing the cells. The cells may either be incubated with the permeabilising agent before freezing, such that they are permeabilised when frozen, or they may be frozen immediately, such that permeabilisation continues during the freezing and/or thawing processes.

The permeabilised cells may be used in any assay which requires one or more key-components of the assay medium to pass into the interior of the cell, e.g. to the cytosol or an intracellular organelle or structure. The key component may be required for a particular reaction to occur (e.g. it may be a substrate or co-factor for that reaction), or may be required to measure the progress of a particular reaction (e.g. an indicator or reporter molecule of some kind).

Also possible, although less common, is that an assay may require a key cellular component to be able to pass into the assay medium, e.g. in order to prevent its concentration increasing substantially within the cell as a result of a given reaction, possibly thereby inhibiting that reaction. Thus, although the following will refer exclusively to a key component of the assay medium, this term should be construed to encompass cellular components which are required to pass out of the cell into the assay medium.

Typically, the component in question will be substantially unable to cross the cell's plasma membrane at the concentration used in the assay. Therefore the cells must be permeabilised so that the component to be able to pass through the membranes of enough of the cells in the preparation for the assay to give meaningful results.

Use of permeabilised cells has the advantage of preserving the organelles etc. in a physiological configuration. The minimal disruption of the cell's structure and biochemistry means that the results from the final assay are more relevant to the intact cell than those from a vesicular assay, and consequently can be relied on confidently.

The cells are typically mammalian cells, and are preferably myocytes (i.e. muscle cells), more preferably cardiac myocytes (including both ventricular and atrial myocytes) or skeletal myocytes. However the technique is suitable for use with any nucleated cell (e.g. any mammalian cells except red blood cells).

The permeabilising agent is preferably selective for the plasma membrane over the intracellular organelle membranes. This allows it to permeabilise the plasma membrane of the cells without significantly permeabilising the membranes of the intracellular organelles such as the nucleus, endoplasmic or sarcoplasmic reticulum, or mitochondria. Preferably the organelle membranes are left intact and substantially not permeabilised at all. Further, the plasma membrane should be left sufficiently intact that the overall structural integrity of the cell is maintained, e.g. organelles such as mitochondria are retained within the cell.

In order to achieve this selective permeabilisation, the permeabilising agent may bind a membrane component of which the plasma membrane has a higher content than the organelle membranes. For example, it may bind to cholesterol, as the plasma membrane has a higher cholesterol content than organelle membranes. Suitable permeabilising agents include saponins, with beta-escin being particularly preferred. These molecules precipitate cholesterol, thus removing it from the membrane and increasing its permeability. Other permeabilising agents may also be used, including various bacterial toxins, such as streptolysin-O and alpha-toxin from Staphylococcus aureus.

The present inventors have found that these permeabilising agents are effective at permeabilising the cell's plasma membrane over a relatively narrow range of concentrations. For example, in preferred embodiments beta-escin is added to a concentration of between 1 and 100 mg/ml, more preferably between 1 and 50 mg/ml. At 1 mg/ml, only about 10% of a sample of cardiac myocytes will be permeabilised, so more preferably a concentration of between about 5 and 20 mg/ml will be used, more preferably about 10 mg/ml.

Preferably at least 50%, 60%, 70%, 80%, 90%, 95%, 96%, 97%, 98% or 990 of the cells should be permeabilised. In preferred embodiments at least 90% of the cells should be permeabilised, and preferably approximately 100%.

Thus the required proportion of the cell population is sufficiently permeable that the key component of the assay medium has access to the cytosol of those cells. It is generally sufficient to regard each cell as either permeable or not permeable to the key component(s) of the assay medium, and to determine the proportion of cells within the population as a whole which can be said to be permeable.

The concentration of permeabilising agent required to achieve the required degree of permeabilisation may vary depending on the commercial source or batch of the permeabilising agent, or the type of cells to be permeabilised.

The skilled person will be able to determine an appropriate concentration of permeabilising agent to use in any particular case. For example, a population of the relevant cell type may be contacted with one or more concentrations of the permeabilising agent, preferably a range of concentrations. The cell population is also contacted with a test agent whose presence within the cell or transit across the membrane can be measured. The proportion of the cell population which displays permeability of the plasma membrane to the test agent at each concentration of the permeabilising agent may then be determined. The minimum concentration of the permeabilising agent which provides the required degree of cell permeabilisation may then be used in the preparation of the frozen cell stock.

The test agent may be any molecule which is substantially unable to cross an intact cell membrane (i.e. is substantially unable to cross the membrane in the absence of the permeabilising agent) and whose presence within the cell or transit across the cell membrane is detectable. Examples include dyes and other molecules whose presence within cells is detectable visually, spectrophotometrically, or by other suitable means. Examples include trypan blue and fluorescent dyes. Calcium ions are not normally able to cross the plasma membrane from the surrounding medium, but passage of calcium into the cell may be detected by a reporter molecule whose spectrophotometric properties (e.g. fluorescence properties) change on binding calcium, such as Fluo-3.

Alternatively, the test agent may cause a detectable change in cell morphology or other physiologically detectable change in the cell. For example, calcium ions cause myocytes to contract from a rod-like morphology to a more spherical morphology. This change may be used to determine the proportion of permeabilised myocytes in a given population.

The Appendix below provides examples of the use of trypan blue and calcium ions in assays to determine effective concentrations for particular permeabilising agents in relation to rabbit cardiac myocytes. The skilled person will be able to adapt that teaching to any desired cell type and assay system.

The permeabilising agent may be rendered substantially ineffective or inactivated by an appropriate dilution of the thawed cell preparation before performing an assay. A dilution of about ten-fold is preferred. This would be applicable, for example, to a preparation of cardiac myocytes having a starting concentration of beta-escin of about 10 mg/ml.

Thus, a dilution may be carried out directly on the thawed cell preparation or as the preparation thaws. In other words, a dilution may be carried out on the just thawed or thawing cell preparation without any procedures or manipulations being carried out on the preparation first, i.e. without any intervening procedures or manipulations.

For example, a diluent may be added directly to the just thawed cell preparation, or directly to the partially frozen or completely frozen cell preparation, which may assist thawing.

Accordingly, an assay may be performed on such a diluted cell preparation, alternatively to being performed on an undiluted thawed cell preparation.

It will be seen that this greatly simplifies the preparation of the eventual assay, because no complicated procedures are necessary to remove or inactivate the permeabilising agent before beginning the assay.

Thus, it is not necessary for cells to be isolated from other component(s) of the cell preparation before use in an assay. For example, it is not necessary for cells to be collected and washed in a suitable medium followed by resuspension in an assay medium for the assay. Therefore, an assay may be performed directly on a just thawed cell preparation or a diluted cell preparation without any procedures or manipulations being carried out on the preparation first, i.e. without any intervening procedures or manipulations, as described in more detail below.

The cell density in the frozen preparation can be chosen according to the dilution required to inactivate the permeabilising agent, as well as the total number of cells and the cell density required for the assay. For example, an assay may require a density of approximately 10⁵, 10⁶ or 10⁷ cells per ml. Thus the frozen cell preparation may have a cell density of 10⁶, 10⁷ or 10⁸ cells per ml so that a 10-fold dilution provides the required density in the assay. For example, in the examples described below, the frozen cell preparation has a density of 1×10⁷ cells per ml, which is diluted to 1×10⁶ cells per ml to perform SR calcium flux assays.

The frozen cell preparation is preferably stored below −70° C., more preferably below about −80° C. At temperatures of −80° C., sarcoplasmic reticulum activity in a preparation of permeabilised cardiac myocytes can be maintained for 6 months or more.

The pH of the cell preparation is typically buffered between about pH 6.8 and about pH 7.4. This will normally be achieved by inclusion of an organic buffer, such as Tris, or any of the “Goods buffers”, including HEPES, BES, TES or PIPES.

The identity and concentration of the buffer may depend on the use to which the cell preparation is to be put after thawing. In general, cellular metabolism has an acidifying effect on the solution. As a result, it is desirable to use at least buffers at a concentration of about 5 mM or more, preferably about 25 mM, but up to about 50 mM if required. The buffer may be present at up to 100 mM if it has an anionic residue (e.g. HEPES), in which case the anionic buffer may be present as the major anion in the solution.

It will be recognised that other components of the system may also have buffering activity.

The cell preparation typically also comprises background electrolytes. Preferably the preparation comprises one or more monovalent metal cations, such as potassium and/or sodium ions, along with one or more suitable, non-toxic anion. Preferably potassium ions are present at about 100 mM, but may range in concentration from about 50 mM to about 200 mM. Sodium ions, if present, are preferably at from about 5 mM to about 40 mM.

Positively charged species are typically not present in total at more than about 300 mM. Likewise, negatively charged species are typically not present in total at more than about 300 mM. Thus the concentrations of electrolytes may be chosen depending on the concentations of other charged species in the solution, such as the buffer (see above), chelating agents, etc.

Anionic electrolytes may include ions such as chloride, preferably present at about 30 mM to about 40 mM, as well as aspartate, glutamate, methyl-sulphate or any other suitable anion. The skilled person will be capable of selecting suitable electrolytes and determining suitable concentrations thereof.

Other components may be present in the system depending on the intended use of the cell preparation when thawed. Many of these components could be added to the cell preparation after thawing, but may alternatively be present in the frozen cell preparation to minimise the number of different components to be added after thawing the cell preparation, and so reduce complexity of manipulation.

For example, it may be desirable that the cells contain substantially no free calcium ions. This may be achieved by washing the cells after permeabilisation. Additionally or alternatively a calcium-specific chelating agent may be added to the solution before or after freezing. A suitable agent is EGTA. Any suitable salt may be used, although a potassium salt is preferred.

A calcium-specific chelating agent may be particularly useful where the cells are to be used for an assay involving calcium flux, such as a sarcoplasmic reticulum calcium flux assay. Typically exogenous calcium will be added to the cells after thawing to perform such an assay. Preferably the concentration of chelating agent is such that it chelates all the endogenous calcium ions present in the cells, but does not unduly affect the concentration of any such exogenously added calcium. For a cell preparation which is to be diluted 10-fold prior to the assay, a suitable concentration (e.g. of EGTA) is up to about 0.1 mM. Preferably the concentration is about 0.05 mM. Other calcium chelating agents may be used; the concentration used may be adjusted in order to give similar chelation to any particular amount of EGTA.

Cell preparations to be used in assays which rely on an ATPase activity may contain ATP. Examples of such assays include those relying on calcium uptake by the SR, which is mediated by the sarco-endoplasmic reticulum ATPase (SERCA). Typically at least 1 mM ATP will be present in a cell preparation which is to be diluted 10-fold prior to commencement of the assay. Preferably about 5 mM ATP is present. However less, e.g. about 0.3 mM to 0.5 mM ATP may be present if the assay solution also contains creatine phosphate (see below).

Any salt of ATP may be used, although the sodium salt is preferred. The magnesium salt is preferably not used, because Mg²⁺-ATP is the substrate for the SERCA. The operator can therefore control precisely when the assay is to begin by adding exogenous magnesium ions to the system. Thus if the frozen cell preparation is to be used for a calcium flux assay, the cell preparation is preferably kept substantially free of magnesium ions. This may be achieved by including EDTA or another Mg²⁺ chelating agent in the cell preparation.

Creatine phosphate may be present in the cell preparation to regenerate ATP from ADP. This may help to prevent ADP formed (by hydrolysis of ATP) during an assay from inhibiting an ATPase. Preferably between 3 mM and 20 mM creatine phosphate is present in a solution to be diluted 10-fold before commencement of an assay. More preferably, a concentration of between about 10 mM and about 15 mM is used.

In an assay for measuring calcium uptake by an organelle (e.g. the SR), it may be desirable to include an agent capable of precipitating calcium from solution at a suitable concentration of calcium. A suitable precipitting agent will form a precipitate with calcium in the organelle and hence prevent the free calcium concentration in that organelle from achieving levels at which influx of calcium is inhibited. Therefore the precipitating agent should form precipitates at a calcium concentration above that of the assay medium, in order, not to interfere with the calcium ions outside the relevant organelle. Oxalate is one example. Fluorides and phosphates should be avoided, because of the toxicity of fluorides, and the further biological activities associated with phosphate ions.

A concentration of oxalate of approximately 20 mM in the final assay buffer will maintain the calcium solution in the SR of a myocyte preparation at below about 10 μM. A concentration of between about 5 mM and about 30 mM oxalate may therefore be used in a cell preparation. For cell preparations which are to be diluted prior to an assay it is preferred not to concentrate the oxalate in the cell preparation, but instead to use a diluent having the same concentration of oxalate.

Any suitable salt may be used, the potassium salt being preferred.

To minimise the effects of endogenous protein kinase activity on the target proteins (e.g. SERCA, Plb, RyR) of the assay, one or more protein kinase inhibitors may be included in the cell preparation. For example, it may be desirable to include an inhibitor of cAMP-dependent protein kinase. Additionally or alternatively, where the cell preparation is to be diluted prior to an assay, the protein kinase inhibitor can be added to the diluent. A preferred kinase inhibitor is H89 (source), which is preferably used at 5-50 W, more preferably 10-30 μM, more preferably approximately 20 μM. H89 (N-[2-(p-Bromocinnamylamino)ethyl]-5-isoquinoline sulfonamide, which is readily commercially available) is particularly effective against cAMP-dependant kinase in particular. However, in certain assays, it may be desirable to preserve the kinase activity of the preparation, in which case kinase inhibitors can be omitted.

Typically substantially no cryoprotectant is present in the cell preparation. These agents are conventionally used in the cryopreservation of proliferating cells to prevent the cells from rupturing. However, such components are unnecessary in the preparations of the present invention in which it is necessary for the cells to be permeabilised, having been thawed. Commonly used cryoprotectants are glycerol and DMSO. Others include ethylene glycol, propylene glycol, hydroxy-ethyl-starch, polyvinylpyrrolidone and polyethlylene oxide.

Indeed, results described herein using cell preparations of the invention show that activity of organelles and other intracellular structures is preserved when freezing cell preparations in the absence of conventional cryoprotectant, such as glycerol or DMSO (see, for example, Table 1). This is surprising in view of the known difficulty with cryopreserving organelles in a fully functionally intact state.

Thus, in preparing a cell preparation of the invention there may be substantially no cryoprotectant, for example, glycerol or DMSO, present, such that there may be substantially no cryoprotectant present in a cell preparation of the invention.

For example, a cell preparation of the invention may contain cells, for example mycocytes, such as cardiac myocytes; a permeabilising agent, for example a saponin, such as beta-escin; a buffer, buffering the preparation between about pH 6.8 and pH 7.4, for example an organic buffer, such as HEPES at pH 7.0; background electrolytes, for example a source of monovalent metal cations, such as potassium ions, for example KCl; and substantially no cryoprotectant, such as glycerol or DMSO.

A cell preparation of the invention may contain cells, for example mycocytes, such as cardiac myocytes; a permeabilising agent, for example a saponin, such as beta-escin; a buffer, buffering the preparation between about pH 6.8 and pH 7.4, for example an organic buffer, such as HEPES at pH 7.0; background electrolytes, for example a source of monovalent metal cations, such as potassium ions, for example KCl; a calcium-specific chelating agent, for example EGTA; ATP; creatine phosphate; a low affinity Ca²⁺ precipitating agent, for example oxalate; and substantially no cryoprotectant, such as glycerol or DMSO.

Accordingly, as described above, it is not necessary for cells to be isolated from other component(s) of the cell preparation before use in an assay. For example, it is not necessary for cells to be collected and washed in a suitable medium followed by resuspension in an assay medium for the assay. Therefore, an assay may be performed directly on a just thawed cell preparation or a diluted cell preparation without any procedures or manipulations being carried out on the preparation first, i.e. without any intervening procedures or manipulations as described in more detail below.

This greatly simplifies the preparation of the eventual assay in which the cell preparation is used because no complicated procedures are necessary to remove or inactivate a cryoprotectant before beginning the assay.

In a further aspect, the present invention provides a frozen cell preparation as prepared by the methods described herein.

In a further aspect, the present invention provides a method of determining an activity of a target intracellular component of a permeabilised cell, the method comprising providing a frozen cell preparation of the invention, thawing said cell preparation, and performing an assay for the activity of the target component.

In preferred embodiments, the invention provides a method of determining an effect of a test substance on an activity of a target intracellular component of a permeabilised cell, the method comprising providing a frozen cell preparation of the invention, thawing the cell preparation, contacting the thawed, permeabilised cells with said test substance, and performing an assay for the activity of the intracellular structure or organelle.

The target component may be a component (e.g. a protein) associated with an intracellular structure or organelle present in the relevant type of permeabilised cell. For example, a preparation of permeabilised myocytes may be used to study myofibrils (contractile proteins), nuclei, mitochondria, or sarcoplasmic reticulum components.

In the instance of the SR, the change in activity is determined by detecting changes in Ca²⁺ levels following contact between the heart cell and the substance under test. Within the sarcoplasmic reticulum, the protein in question may be the Ca²⁺ ATPase (SERCA), phospholamban (Plb), ryanodine receptor (RyR), FKBP12.6, Sorcin, Calmodulin, Ca-calmodulin-activated kinase, or cAMP-activated kinase.

The assay conditions can be easily adapted to study specific protein targets. This is because regulatory proteins are still retained within the system, thus allowing the assay to include test agent interaction with these auxiliary proteins.

The cell preparation may be contacted with a diluent prior to beginning the assay, to form an assay mixture. The diluent may perform any or all of a number of roles. It may dilute the permeabilising agent in the previously-frozen cell preparation to a concentration at which it is substantially ineffective. It may dilute the cells of the cell preparation to the required cell density for the assay to be performed. It may adjust the concentration of other components in the previously-frozen cell preparation to the required value for the assay. It may provide components not present in the frozen cell preparation which are required to perform the assay. The frozen cell preparation may be thawed prior to addition of the diluent, or the diluent may be added to partially or completely frozen cells in order to assist the thawing process.

Thus, a diluent may be added directly to the thawed cell preparation or as the preparation thaws. In other words, a diluent may be added to the just thawed or thawing cell preparation without any procedures or manipulations being carried out on the preparation first, i.e. without any intervening procedures or manipulations. For example, a diluent may be added directly to the just thawed cell preparation, or the partially frozen or completely frozen cell preparation.

Thus, where a cell preparation contains ten times the concentration of particular components required for a given assay, the cell preparation may be contacted with nine volumes of a diluent lacking those components.

Other components of the diluent (e.g. buffers, electrolytes, etc.) may also be present in the cell preparation. In preferred embodiments, any components present in both cell preparation and diluent are equimolar in the two solutions.

In preferred embodiments the diluent does not contain permeabilising agent, and is used to dilute the permeabilising agent in the cell preparation to a level at which it is substantially inactive or ineffective.

Accordingly, an assay may be performed on such a diluted cell preparation or assay mixture, alternatively to being performed on an undiluted thawed cell preparation.

In particular, an assay may be performed directly on any of these cell preparations. In other words, an assay may be performed on any of these cell preparations without any procedures or manipulations being carried out on the preparation first, i.e. without any intervening procedures or manipulations. For example, an assay may be performed directly on the diluted cell preparation, or the just thawed cell preparation.

It will be seen that this greatly simplifies the preparation of the assay, because no complicated procedures are necessary before beginning the assay. Thus, it is not necessary for cells to be isolated from other component(s) of the cell preparation before use in an assay. For example, it is not necessary for cells to be collected and washed in a suitable medium followed by resuspension in an assay medium for the assay. Therefore, an assay may be performed directly on a just thawed cell preparation or a diluted cell preparation without any procedures or manipulations being carried out on the preparation first, i.e. without any intervening procedures or manipulations. As will be appreciated by those skilled in the art, intervening procedures or manipulations which are avoided relate to preparation of the cells to make them ready for use in an assay, for example, to remove compounds the presence of which may be detrimental in an assay. Procedures or manipulations appropriate to the particular assay being carried out may be performed, for example, adding a test substance the effect of which is to be assessed in an assay as described above, or adding an initiating agent to start the assay as described below.

Thus, the method may comprise thawing the cell preparation and performing an assay for the activity of the target component directly on the thawed cell preparation, without first isolating the cells from other component(s) of the cell preparation, for example by washing and resuspending the cells.

The method may comprise thawing the cell preparation, contacting the cell preparation with a test substance and performing an assay for the activity of the target component, to determine the effect of the test substance on the activity of the target component, directly on the cell preparation, without first isolating the cells from other component(s) of the cell preparation, for example by washing and resuspending the cells.

The method may further comprise contacting the cell preparation with a diluent to form an assay mixture and performing an assay for the activity of the target component, which may be to determine the effect of a test substance on the activity of the target component, directly on the assay mixture (diluted cell preparation), without first isolating the cells from other component(s) of the cell preparation, for example by washing and resuspending the cells.

The method may comprise adding one or more initiating agents in order to begin the assay. The initiating agent may be a substance which is required for activity of the target component. The initiating agent may, for example, be a substrate for the target component, or for a component upstream of the target component in a reaction pathway or cascade. The initiating agent may be added as part of the diluent, or separately, e.g. after the cell preparation and diluent have been incubated together for a period of time to allow complete thawing of the cells.

For example, in an assay dependent on SERCA activity, the initiating agent may be magnesium ions. As explained above, these can combine with ATP already present in the assay mixture to form Mg²⁺-ATP, the substrate for the SERCA.

Other components may be added, in the diluent or separately, e.g. inhibitors of components whose activity would affect the assay. For example, an assay to monitor Ca²⁺ release from the SR may make use of a Ca²⁺-ATPase inhibitor, such as thapsigargin. Inhibition of the ATPase will prevent released calcium ions from being taken back up into the SR. Thus the rate of accumulation of calcium ions observed in the medium will provide a direct indication of the rate of release from the SR. Likewise, an assay for calcium uptake by the SR may utilise inhibitors of calcium release from the SR, such as ruthenium red. Any calcium flux assay will typically use an indicator for the presence of calcium ions, e.g. an agent capable of producing a detectable signal on contact with calcium. Various suitable fluorescent dyes are known, including Fluo-3. Such components may also be present in the cell preparation, if desired.

Thus, in preferred embodiments, a complete assay system can be set up by simple addition of an aliquot of diluent to an aliquot of frozen cells, optionally followed by a single addition of an initiating agent to the resulting assay mixture. This simplicity is particularly appropriate for medium/high-throughput assays, and/or automated assays.

In a further aspect, the present invention provides a kit comprising a frozen cell preparation as prepared by the methods described herein, in combination with a diluent as described in relation to the methods above. Optionally the kit comprises instructions for performing an assay according to any, appropriate aspect of the invention.

It will be apparent from the foregoing that it is also possible to permeabilise the cells after thawing. Thus, in a variation of earlier aspects of the invention, there is also provided a method for determining an activity of an intracellular structure or organelle of a permeabilised cell, the method comprising providing a frozen cell preparation, thawing said cell preparation to produce a thawed cell population, contacting the thawed cell population with a permeabilising agent to produce a population of permeabilised cells, and performing an assay for the activity of the intracellular structure or organelle.

Also provided is a method for determining an effect of a test substance on an activity of an intracellular structure or organelle of a permeabilised cell, the method comprising providing a frozen cell preparation, thawing said cell preparation to produce a thawed cell population, contacting the thawed cell population with a permeabilising agent to produce a population of permeabilised cells, contacting said permeabilised cells with said test substance, and performing an assay for the activity of the intracellular structure or organelle.

Preferred features of these methods are as described above in relation to earlier aspects of the invention. In particular, in these variations an assay may be performed directly on the permeabilised cells, as described above.

The following illustrates a use of the permeabilised cell preparations of the invention. The present inventors have found that permeabilised myocytes can maintain Ca²⁺ uptake capacity for many hours and can be used in conjunction with a fluorescent Ca²⁺ indicator thereby allowing the use of standard spectrometer equipment as a convenient and inexpensive way to measure Ca²⁺ uptake.

A preparation of frozen cardiac myocytes may therefore be provided according to the invention for analysis of sarcoplasmic reticulum calcium flux.

Such a preparation may contain:
a) cardiac myocytes;
b) a source of monovalent metal cations, preferably potassium ions, such as KCl or other background electrolyte;
c) ATP;
d) Creatine Phosphate;
e) organic buffer, e.g. at pH 7.0, such as HEPES;
f) EGTA;
e) low affinity Ca²⁺ precipitating agent, e.g. oxalate;
f) permeabilising agent, e.g. a saponin, such as beta-escin.

With this preparation of frozen cells may be provided one or both of the following diluent solutions:

### Diluent 1:

a) a source of monovalent metal cations, preferably potassium ions, as found in the cell preparation;
b) ATP;
c) Creatine Phosphate;
d) organic buffer as found in the cell preparation;
e) EGTA;
f) low affinity Ca²⁺ precipitating agent as found in the cell preparation, e.g. oxalate;
g) protein kinase inhibitor, e.g. H89.

This diluent is suitable for a SR calcium uptake assay as described in the Examples. An assay mixture is prepared by adding 9 volumes of diluent to 1 volume of cell preparation. All components present in both cell preparation and diluent are equimolar in the two solutions. Each component may have a concentration as set out above, or as in the Examples. Magnesium ions may be added to the assay medium to initiate SERCA activity. These may be present in the diluent, or may be provided separately, in the kit if required, e.g. in the form of MgCl₂.

The diluent and/or the cell preparation may also comprise an agent which provides a detectable signal on contact with calcium ions, e.g. a fluorescent dye such as Fluo-3.

It may also contain one or more agents for inhibiting release of calcium from the SR, such as ruthenium red.

The uptake assay may be used to determine substances under test which a) act directly or indirectly to alter the phosphorylation of phospholamban as this will enhance Ca²⁺ uptake; b) substances that interfere with the interaction between phospholamban and the Ca²⁺ ATPase; c) substances that act directly on the Ca²⁺ ATPase to enhance or inhibit the activity of the enzyme; and d) substances that act on the Ca²⁺ leak from the SR via the Ca²⁺ release channel (ryanodine receptor) or via independent leak pathways.

### Diluent 2:

a) a source of monovalent metal cations, preferably potassium ions, as found in the cell preparation;
b) ATP;
c) Creatine Phosphate;
d) organic buffer as found in the cell preparation;
e) EGTA;
f) low affinity Ca²⁺ precipitating agent as found in the cell preparation, e.g. oxalate;
g) MgCl₂;
h) SERCA inhibitor, e.g. thapsigargin;
i) protein kinase inhibitor, e.g. H89.

This diluent is suitable for a SR calcium release assay as described in the Examples. As with diluent 1, all components present in both cell preparation and diluent are equimolar in the two solutions, and may have concentrations as set out above, or as in the Examples. An assay mixture is prepared by adding 9 volumes of diluent to 1 volume of cell preparation.

The diluent and/or the cell preparation may also comprise an agent which provides a detectable signal on contact with calcium ions, e.g. a fluorescent dye such as Fluo-3.

This assay may be used to determine substances under test which a) act directly or indirectly to alter Ca²⁺ extrusion via the ryanodine receptor; b) substances that interfere with the interaction between the ryanodine receptor and the various modulatory proteins e.g. FKBP12.6, sorcin, c) substances that may alter Ca²⁺ fluxes by changes in the phosphorylation status of the ryanodine receptor; and d) substances that act directly on Ca²⁺ leak pathways that are independent of the ryanodine receptor.

For both the uptake and release assays, the amount of Ca²⁺ in said solution can be measured either by following a time course of the Ca²⁺ levels, e.g. by measuring every 1 to 2 seconds for up to 10 minutes; or by measuring the level of Ca²⁺ at a set time.

These assays can be applied to a multi-well format (e.g. 96 well, 384 well, or more) which is suitable for standard fluorescence plate readers.

In this form, the SR preparation can be used to profile a very large number of candidate compounds. For example, in the context of an uptake assay, a high through-put screen can be used for >10,000 test compounds, to provide a read-out that shows whether a compound stimulates or inhibits SERCA mediated uptake. A medium through-put screen would allow 100-200 compounds to be screened quickly while examining the time course of uptake. This type of read-out would give a more detailed read-out of the test compound's action, i.e. whether the compound increased maximum turn-over rate or the Ca²⁺ sensitivity of Ca²⁺ SERCA-mediated Ca²⁺ uptake.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1: Ca²⁺ ATPase mediated uptake in permeabilised cell preparations.

A. Shows a sample trace of Ca²⁺ concentration within a suspension of 5×10⁵ cells/ml (Fura-2 fluoresence) (SERCA activity assay). As indicated above the figure, Ca²⁺ was added to increase the [Ca²⁺] within the aggregate to ˜1.5 μM. No Ca²⁺ uptake occurs due to the absence of Mg²⁺ (and therefore MgATP). Addition of Me starts the reaction. The initial increase of [Ca²⁺] is caused by displacement of Ca²⁺ from ATP. The SR subsequently takes up the Ca²⁺ within the cuvette, a process that is enhanced by addition of 10 uM cAMP.

B. Shows detailed SERCA parameters derived from complete time course signals.

C. Shows inhibition of SERCA by thapsigargin.

FIG. 2: Caffeine stimulated Ca²⁺ leak from permeabilised cell aggregates

Shows the principle of measuring the Ca²⁺ sensitive leak from cell aggregate preparations. Ca²⁺ uptake is stimulated by addition of an aliquot of Ca²⁺. SERCA inhibition is induced by thapsigargin (5 uM). Ca²⁺ leak is stimulated by the addition of 40 mM caffeine and blocked by addition of ruthenium red (as indicated).

FIG. 3: Concentration-response curve showing permeabilising effect of Calbiochem saponin (0.01-1000 μg/ml) in 5 mM EGTA and 5 mM Ca²⁺ EGTA.

Permeabilisation was quantified using the Ca²⁺-contraction assay and counts were normalised against the control count. Each point is the mean±s.e. mean of 3-8 observations.

FIG. 4: Concentration-response curve showing cell counts in 5 mM EGTA and 5 mM Ca²⁺ EGTA exposed to Sigma saponin at 1-1000 μg/ml. Results were assessed using the Ca²⁺-contraction assay and normalised against the control count. Each point is the mean±s.e. mean of 6 observations.

FIG. 5: Concentration-response curve showing the permeabilising effect of β-escin (0.01-10000 μg/ml) in 5 mM EGTA and 5 mM Ca²⁺ EGTA. Results were quantified using the Ca²⁺-contraction assay and normalised against the control count. Each point is the mean±s.e. mean of 3-10 observations.

FIG. 6: Concentration-response curve showing the permeabilising effect of Calbiochem saponin (0.1-10000 μg/ml) in 5 mM EGTA. Results were quantified using the access of trypan blue and normalised against the control count. Each point is the mean±s.e. mean of 3 observations.

FIG. 7: Concentration-response curve showing the permeabilising effect of β-escin (0.1-10000 μg/ml) in 5 mM EGTA. Results were quantified using the access of trypan blue and normalised against the control count. Each point is the mean±s.e. mean of 3 observations.

FIG. 8: Concentration-response curves for (a) Calbiochem saponin (0.01-10000 μg/ml), (b) Sigma saponin (1-1000 μg/ml) and (c) β-escin (0.01-10000 μg/ml) in 5 mM EGTA and 5 mM Ca²⁺ EGTA. Results were assessed using the Ca²⁺-contraction assay and normalised against the initial count. Each point is the mean±s.e. mean of 3-10 observations.

FIG. 9: Fit of the non-linear logistic curves for each agent and associated assay method.

ED₅₀ values were estimated from this fit and are shown along side each plot. Each point is the mean±s.e. mean of 3-10 observations.

FIG. 10: Ca²⁺ ATPase mediated uptake in permeabilised cell preparations—Example data from assays with test compounds.

Characteristics of Ca²⁺ uptake were analysed in the presence of various test compounds in assays using a multi-well format. Example data achieved with inhibitory and stimulatory compounds are shown.

Table 1: Sustained activity from cell aggregates.

Myocyte aggregates were held in storage for approximately 6 months or more at −80 C using the procedure described below in the Examples. On defrosting the characteristics of Ca²⁺ uptake were analysed in terms of the maximum rate of uptake (Vmax) and the sensitivity of Ca²⁺ uptake (Km). Neither parameter showed significant deterioration over the storage period. Values are expressed as mean+/−SEM.

Table 2: Stock solutions.

This Table shows the volume of each stock solution required to produce the range of concentrations of permeabilising agent in 1 ml of cell suspension.

Table 3: ED₅₀ values.

ED₅₀ values for each agent studied for Ca²⁺-contraction and trypan blue assay methods.

Table 4: Ca²⁺ ATPase mediated uptake in permeabilised cell preparations—pharmacological characterisation.

Frozen cell preparations were used to analyse characteristics of Ca²⁺ uptake in the presence of various stimulators or inhibitors of SERCA. Results for sensitivity of Ca²⁺ uptake (Km) are shown. Values are expressed as mean+/−SEM.

### EXAMPLES

### Materials and Methods

### Cell Isolation

The procedure is based on the principle that cells within the intact heart can be isolated by gradual removal from their associated connective tissue and cellular syncitium. Before isolation, thorough cleaning of perfusion equipment was carried out. 2-3 1 of double distilled water was perfused through the system followed by 1-2 1 of sterile water. New Zealand White rabbits (2-2.5 kg) were given an intravenous injection of 500 U heparin together with an overdose of sodium pentobarbitone (100 mg/kg). The hearts were rapidly excised, weighed and cannulated onto a Langendorff perfusion column via the aorta. The hearts were perfused in a retrograde fashion at a rate of 25 ml.min⁻¹ (37° C.), initially with 150 ml of Krebs Henseleit solution with the following composition (mM): 120 NaCl, 20 HEPES, 5.4 KCl, 0.52 NaH₂, PO₄, 3.5 MgCl₂.6H₂0, 20 Taurine, 10 Creatine, 11.1 Glucose; pH 7.4 with NaOH at 37° C. [Ca²⁺] within this solution is in the order of 6-7 μM due to Ca²⁺ contamination. This solution aids in washing away blood and reduces the probability of clot formation. Taurine may aid in cardio-protection against Ca²⁺ paradox (Chapman & Tunstall, 1983). Thereafter, the hearts were perfused for 1 min with Krebs Henseleit solution supplemented with 1.4 mg ml⁻¹ collagenase (type 1, Worthington Chemical Co.), 0.1 mg ml⁻¹ protease (type XIV, Sigma Chemical Co.) and a further addition of 50 μM CaCl₂ to activate the enzymes. After a time, such that the enzyme solution had fully perfused the heart and equipment the enzyme containing solution was re-circulated for a further 6 min. The heart was then perfused with a 0.1% Bovine Serum Albumin (BSA) Krebs Henseleit solution with the addition of 62 μM CaCl₂. The BSA containing solution provides extra substrate for superfluous enzyme. The atria were removed and discarded whereas the right and left ventricle was kept for experiments.

### Intact Cell Preparation

The chosen ventricle was carefully cut into small pieces and placed into a culture flask containing 20 ml of the 0.1% BSA containing Krebs Henseleit solution described above. This solution also contained 0.125 mM CaCl₂. The suspension was gently shaken for 30-60 min. After the appropriate time the solution containing the cardiomyocytes was removed. The suspension was subjected to gentle centrifugation and the pellet re-suspended into 0.1% BSA Krebs Henseleit solution containing 0.125 mM CaCl₂. Cells were then washed in the following solution: (mM) 100 KCl, 5 Na₂ATP, 10 Na₂Creatine Phosphate, 5.5 MgCl₂, 25 HEPES, 1 K₂EGTA, pH 7.0 (20-21° C.)

### Preparation of Cells for Freezing

Intact cells within the above solution were counted using a haemocytometer and adjusted to a concentration of 1×10⁶/100 ul (i.e. 1×10⁷ cells.ml⁻¹) in intracellular solutions (S1 or S2 depending on the purpose of the assay: S1: (mM) 100 KCl, 5 Na₂ATP, 10 Na₂Creatine Phosphate, 25 HEPES, 0.5 K₂EGTA, pH 7.0 (20-21° C.), 20 oxalate. S2: (mM) 100 KCl, 5 Na₂ATP, 5.5 MgCl₂, 10 Na₂Creatine Phosphate, 25 HEPES, 0.5 K₂EGTA, pH 7.0 (20-21° C.), 20 oxalate.

Finally the 0.1 mg.ml⁻¹ beta-escin (Sigma) was added and the preparation was immediately aliquoted into cryovials (at 1×10⁶ cells) and placed into a ‘Mr Frosty’ container overnight to allow gradual freezing. These were then placed into liquid nitrogen for freezing.

### Unfreezing of Cell Preparation

900 μl of S1 or S2 diluion solution as appropriate was added to the 100 μl of cryovial cell preparation.

### Composition of S1 Dilution Solution

S1: (mM) 100 KCl, 5 Na₂ATP, 10 Na₂Creatine Phosphate, 25 HEPES, 0.05 K₂EGTA, 0.01 K₄Fluo-3, pH 7.0 (20-21° C.), 20 K₄Oxalate, 0.005 Ruthenium Red, 0.05 mM H89.

### Composition of S2 Dilution Solution

S2: (mM) 100 KCl, 5 Na₂ATP, 5.5 MgCl₂, 10 Na₂Creatine Phosphate, HEPES, 0.05 K₂EGTA, 0.01 K₄Fluo-3, 0.005 Thapsigargin, pH 7.0 (20-21° C.), 20 K₄Oxalate, 0.05 mM H89.

### Results

### SR Uptake Assay

(e.g. an assay for SR Ca²⁺ ATPase activity and modulation by phospholamban)

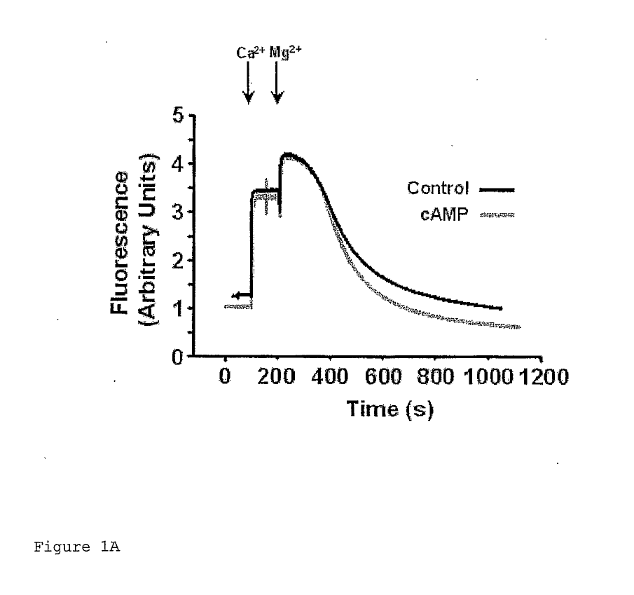

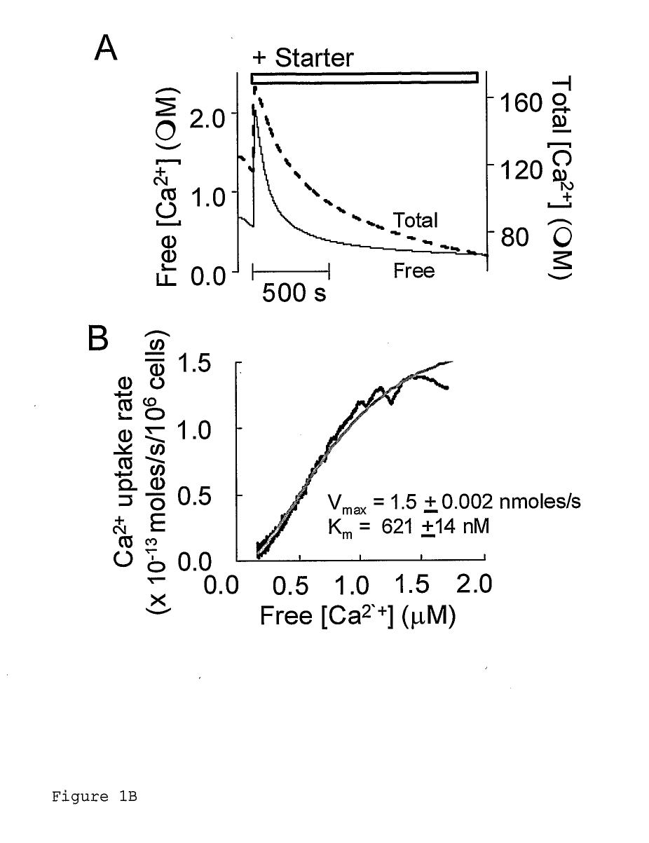

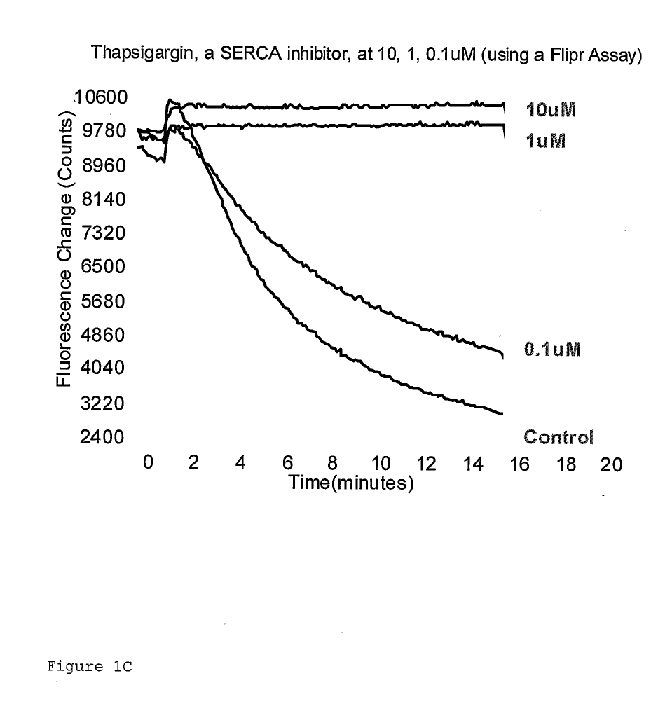

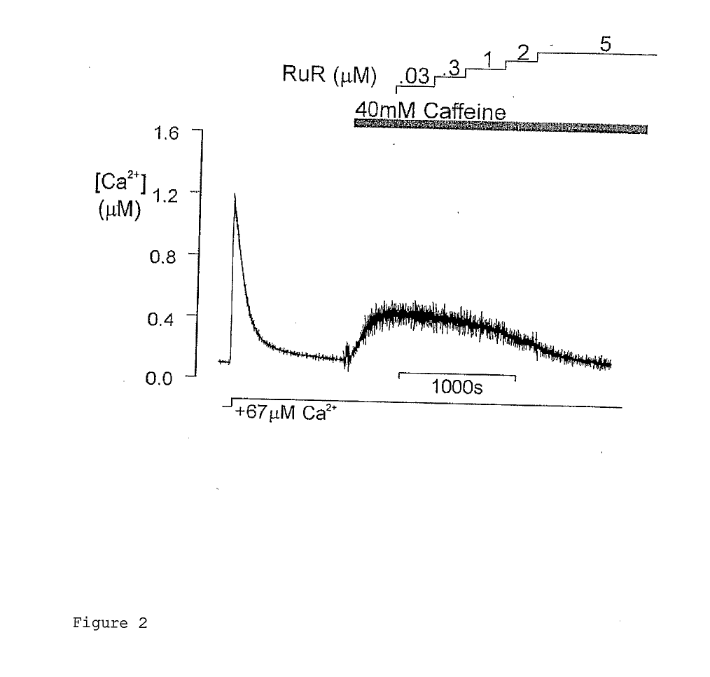

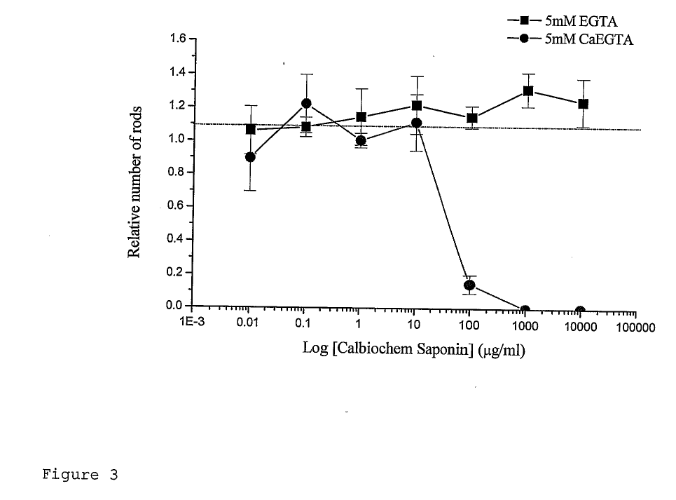

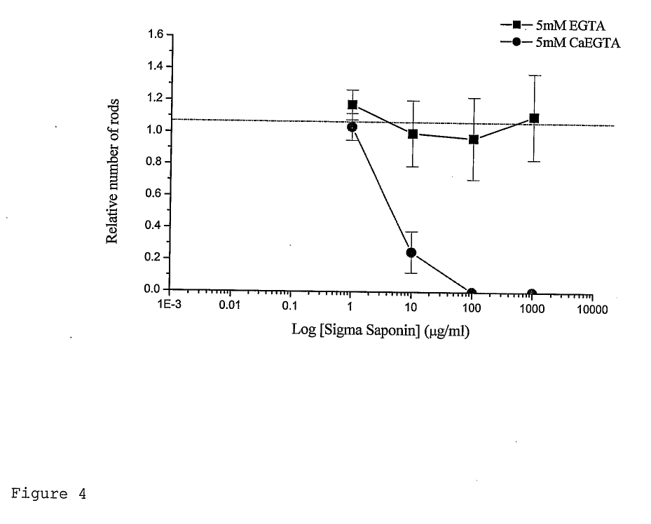

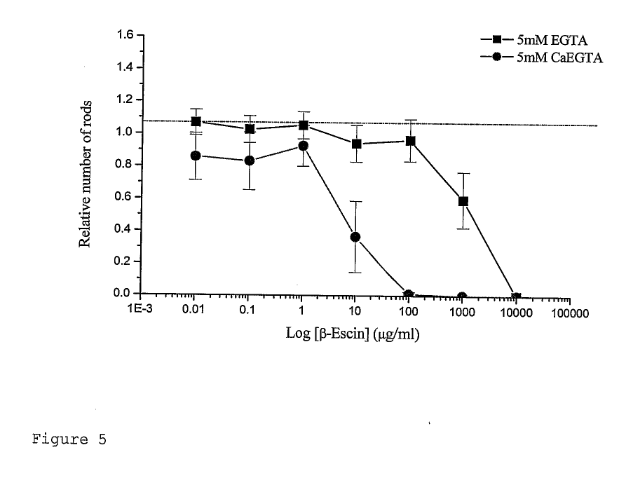

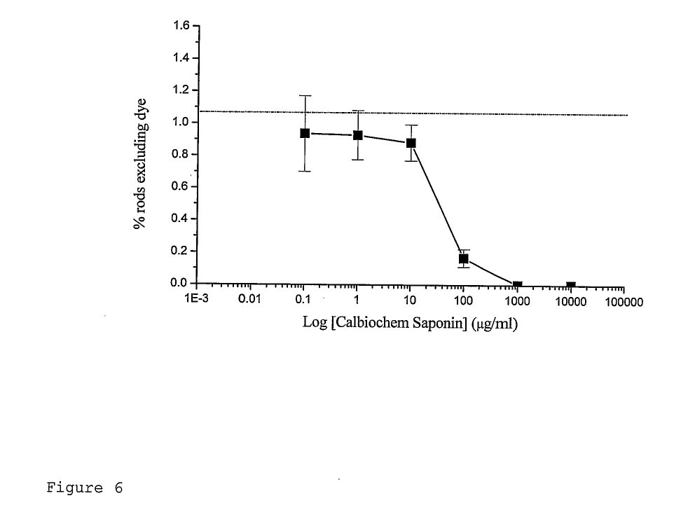

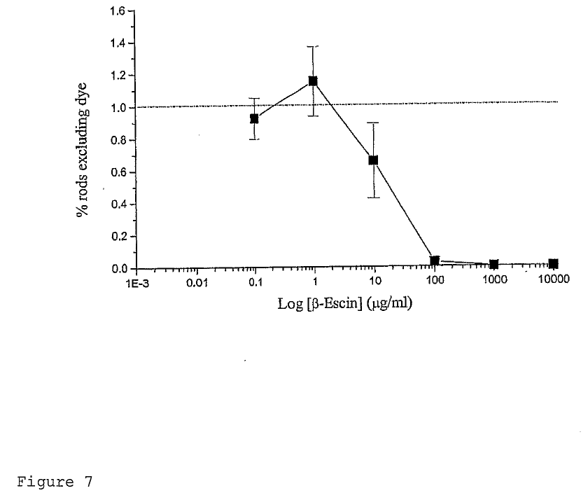

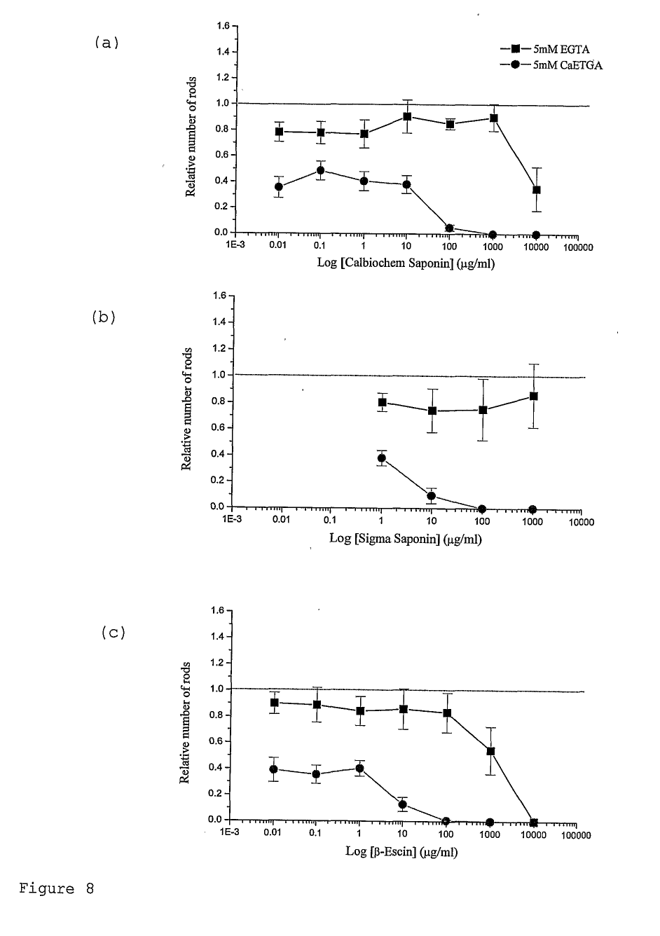

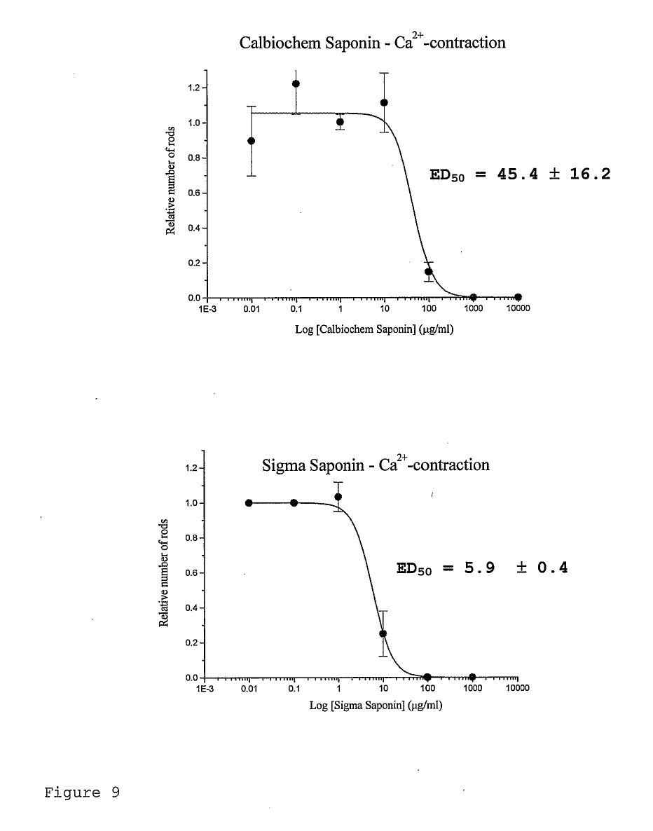

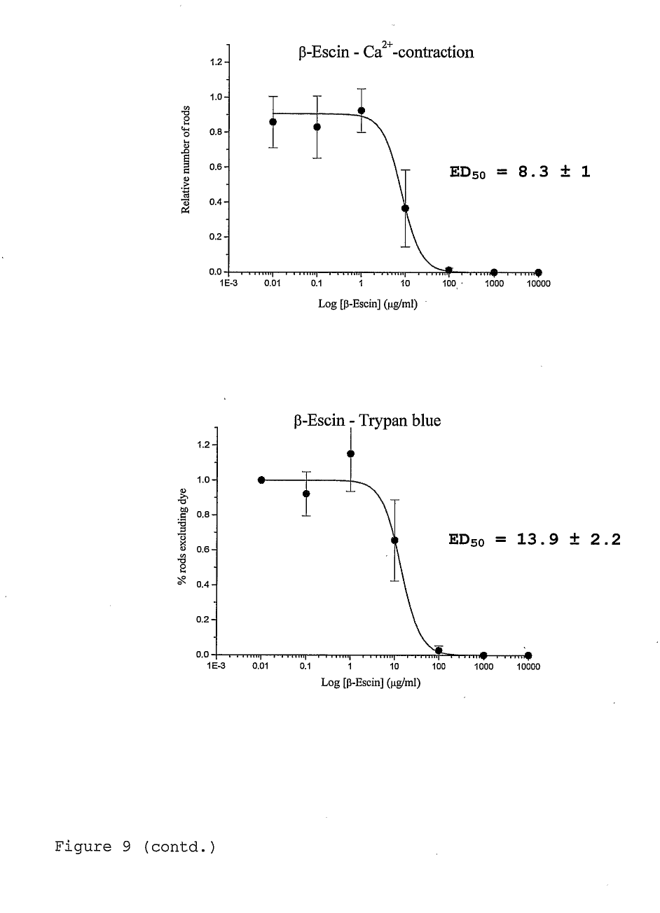

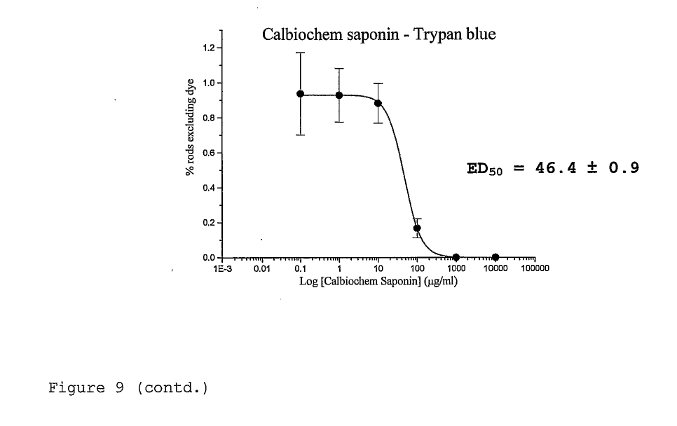

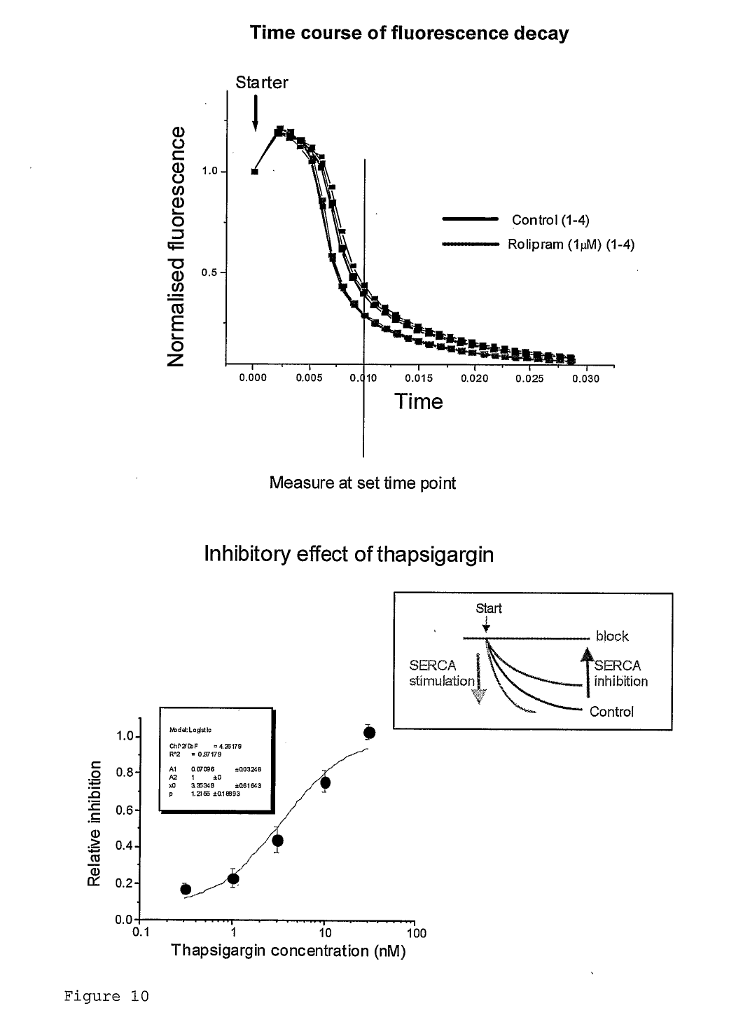

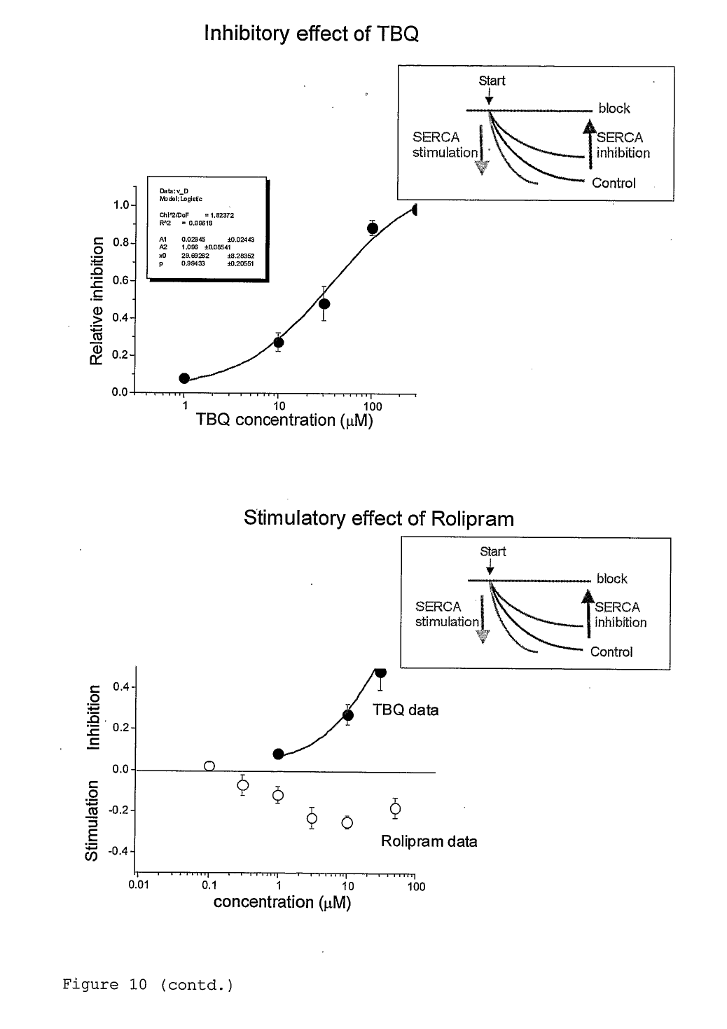

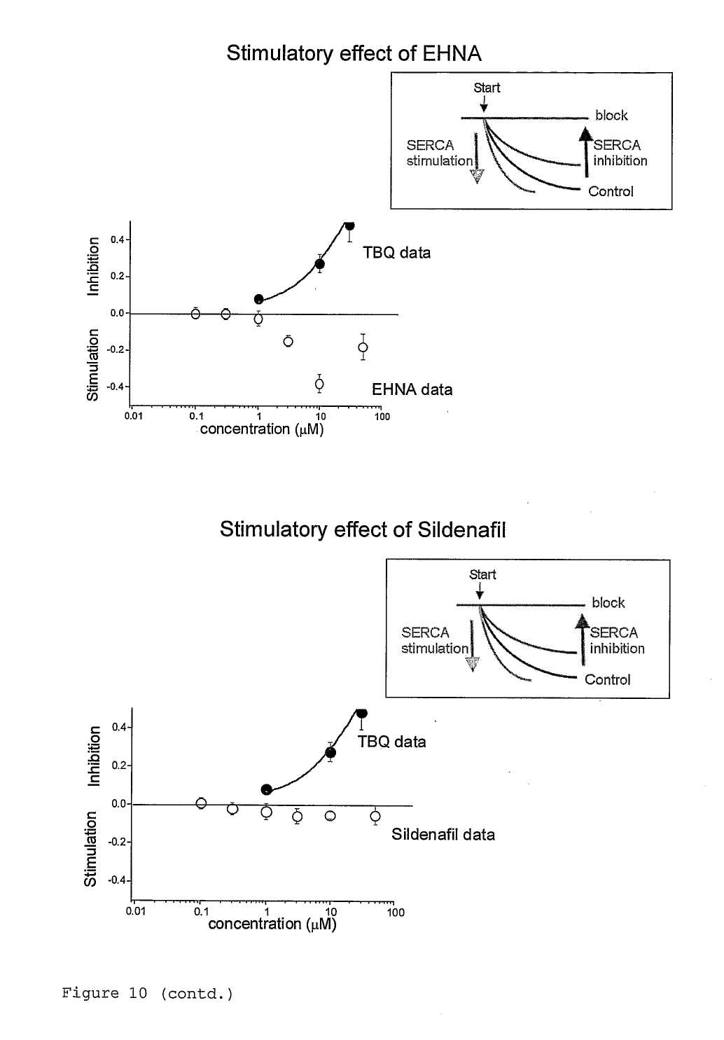

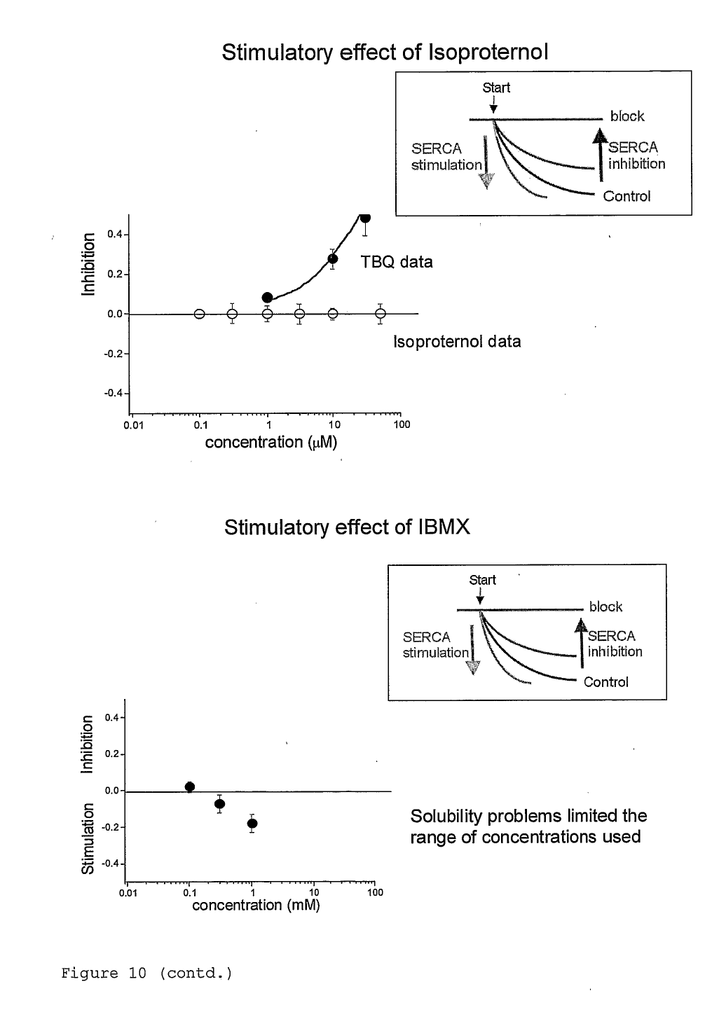

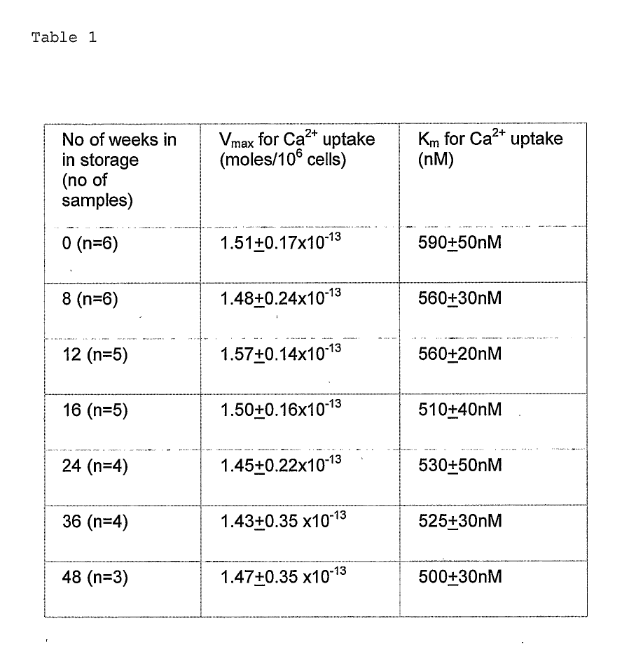

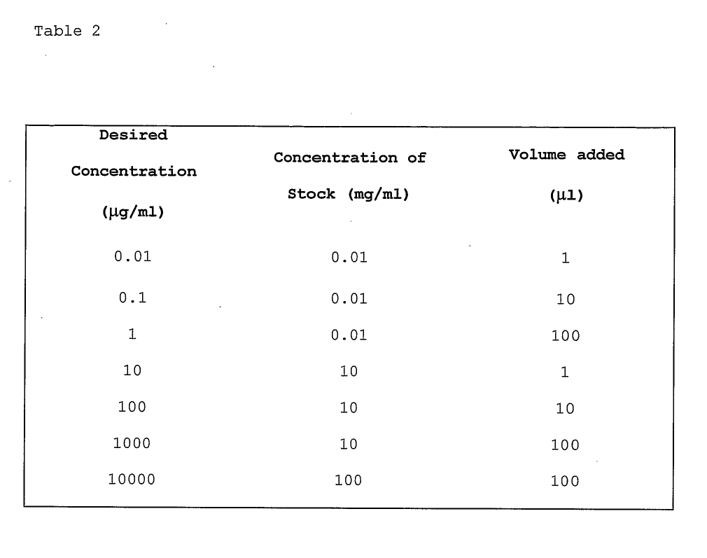

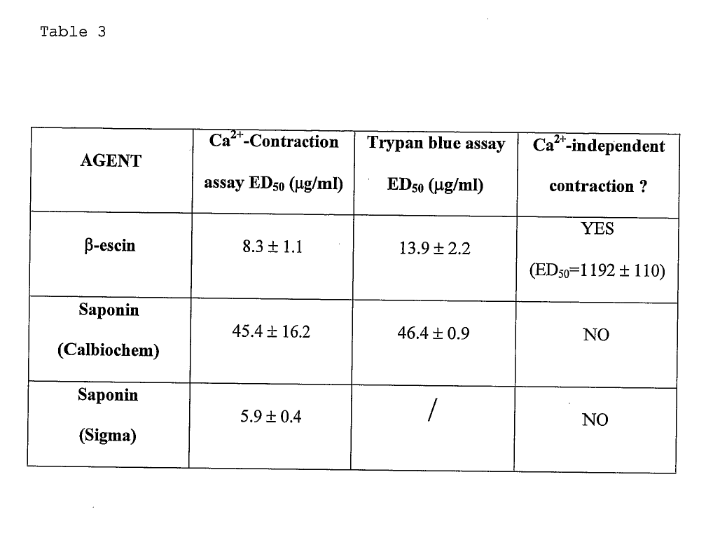

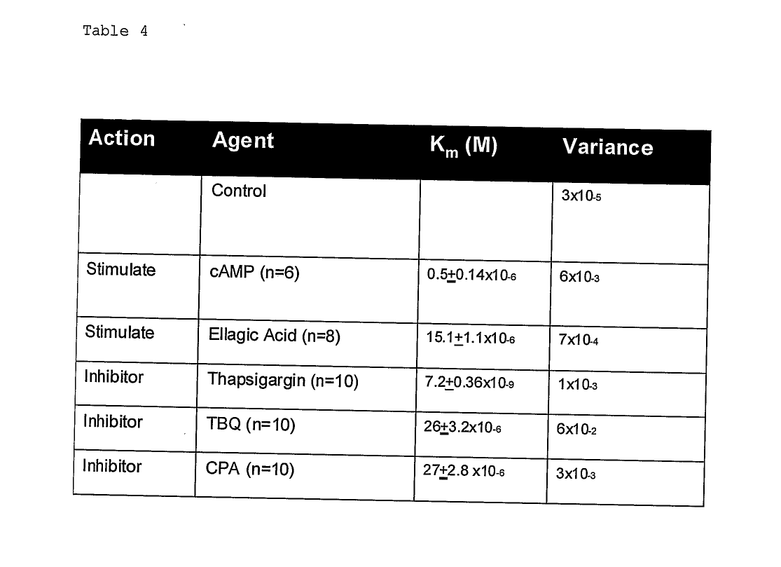

Aliquots of cells are defrosted gradually with the addition of 9 volumes of S1 solution. This was followed by addition of CaCl₂ to increase the free [Ca²⁺] to ˜1.5 μM (˜100 μM total Ca²⁺). The mixture can then be distributed into the 96 or high format arrays. Incubation of the preparation with the compound of interest can occur for 15-20 mins and the SR uptake reaction began by addition of 5.5 mM MgCl₂ (with stirring). This provides the substrate (MgATP) to allow SR uptake of Ca²⁺. There are two options for measurement: (i) follow the time course of the Ca²⁺ uptake profile in detail by measuring every 1-2 s for up to 10 mins (ii) measuring the Ca²⁺ concentration at a set time (e.g. 10 mins) after initiation of the reaction. Compounds that stimulate the SR Ca²⁺ ATPase will cause the decline of Ca²⁺ concentration to occur faster than control (vehicle). Conversely compounds that inhibit the SR Ca²⁺ ATPase will slow the decline. These effects can be monitored in detail (option (i)—see FIG. 1 and description thereof for an example) or by simply measuring Fluo-3 fluorescence at one time point (option (ii)).

### This Assay is Sensitive to:

1. Compounds acting directly or indirectly to alter the phosphorylation of phospholamban, which will enhance Ca²⁺ uptake.
2. Compounds that interfere with the interaction between phospholamban and the Ca²⁺-ATPase.
3. Compounds that act directly on the Ca²⁺ ATPase to enhance or inhibit the activity of the enzyme.
4. Compounds that act on the Ca²⁺ leak from the SR via the Ca²⁺ release channel (ryanodine receptor) or via independent leak pathways.

Note that screening for compounds that interfere with ryanodine receptor leak can be differentiated by using Ruthenium Red (1-5 μM); this compound blocks Ca²⁺ release via the ryanodine receptor. This latter effect can also be detected by a second type of assay configuration (see below).

### SR Ca²⁺ Release Assay

(e.g. to assess the rate of Ca²⁺ loss from the SR both via the ryanodine receptor and by independent routes.)

This assay requires the preparation stored in solution S2 (see above). The cell aggregate preparation is defrosted in 9× volumes of S2 with the addition of 2-3 aliquots of 100 μM CaCl₂. The preparation is then aliquoted into the high format wells (96 or 384 well). The reaction is initiated by addition of a single aliquot of the SR Ca²⁺ ATPase inhibitor thapsigargin (or any other established selective Ca²⁺ ATPase inhibitor). The slow progressive increase in Ca²⁺ concentration within the bath reflects Ca²⁺ loss from the SR both via the ryanodine receptor and via independent leak pathways. Ryanodine receptor mediated leak can be enhanced for purposes of drug screening by inclusion of caffeine (5-10 mM) to the reaction mixture. As with the assay system above, two options for measurements are possible (i) follow the timecourse of the Ca²⁺ release profile in detail by measuring every 1-2 s for 10 mins (ii) measuring the Ca²⁺ concentration at a set time (e.g. 10 mins) after initiation of the reaction. Compounds that stimulate the SR Ca²⁺ leak will cause the increase of Ca²⁺ concentration to occur faster than control (vehicle). Conversely compounds that inhibit the SR Ca²⁺ leak will slow the increase of Ca²⁺ concentration. These effects can be monitored in detail (option (i)—see FIG. 2 and description thereof for an example) or by simply measuring Fluo-3 fluorescence at one time point (option (ii)).

### This Assay is Sensitive to:

1. Compounds acting directly or indirectly to alter Ca²⁺ extrusion via ryanodine receptor.
2. Compounds that interfere with the interaction between the ryanodine receptor and the various modulatory proteins e.g. FKBP12.6, sorcin.
3. Compounds that may alter Ca²⁺ extrusion by changes in the phosphorylation status of the ryanodine receptor.
4. Compounds that act directly on Ca²⁺ leak pathways that are independent of the ryanodine receptor.

Note that screening for compounds that interfere with ryanodine receptor leak can be differentiated by using Ruthenium Red (5-10 μM) this compound blocks Ca²⁺ release via the ryanodine receptor.

Permeabilised myocytes prepared as described above retain significant activity even after 6 months or more at −80° C. On defrosting the characteristics of Ca²⁺ uptake were analysed in terms of the maximum rate of uptake (Vmax) and the sensitivity of Ca²⁺ uptake (Km). Neither parameter showed significant deterioration over the storage period. Values are shown in Table 1, expressed as mean+/−SEM.

### APPENDIX

### Determination of Concentrations for Permeabilising Agents

Commercial preparations of permeabilising agents such as saponins (including beta-escin) may vary significantly in activity between sources and batches. For this reason, it may be desirable to test each new batch of permeabilising agent in order to determine a suitable concentration for any given assay.

The following description illustrates two methods which were used to test permeabilisation of rabbit myocytes by a batch of beta-escin, and to compare two different commercial batches of saponin.

### Methods

### Preparation of EGTA and Ca²⁺ EGTA Solutions

Permeabilisation of myocytes occurred in mock intracellular bathing solutions. This ensured that there were no major changes in intracellular ions and metabolites once the sarcolemma was made hyperpermeable.

Two HEPES-buffered Krebs-based bathing solutions were produced; one with high calcium (Ca²⁺ EGTA), the other with low calcium (EGTA). The two solutions had the same basic composition (mM): KCl 100 (BDH Laboratory Supplies, Poole, England; lot no: A154338 907), NaCl 10 (Sigma-Aldrich Chemicals, Germany; lot no: 01500), MgCl 5.5 (BDH Laboratory Supplies, Poole, England; lot no: 39627), HEPES 25 (Sigma Chemical Company, St. Louis, USA; lot no: 90K5406), ATP 5 (disodium salt; Sigma Chemical Co., St. Louis, USA; lot no: 100K7051), creatine phosphate 10 (disodium salt tetrahydrate; Fluka Chemicals; lot no: 399292/1 40201) and either EGTA or Ca²⁺ EGTA both 5 (Sigma Chemical Co., St. Louis, USA: lot no: 119H5433).

The volume was made up to 100 mls using distilled water and pH was adjusted to exactly 7.0. 20 mls of each solution was pipetted into individual labelled plastic containers (to prevent reaction of ATP with glass). These were frozen (approximately −80° C.) and stored until required. The calcium concentrations of 5 mM EGTA and 5 mM Ca²⁺ EGTA solutions were calculated at <1 nM and 20 μM respectively.

### Cell Counting

The haemocytometer method of cell counting was used to count the number of non-permeable myocytes before and after application of the permeabilising agent (either saponin or β-Escin). Freshly dissociated cardiac muscle cells were obtained in a solution of low calcium. These were filtered and transferred into two 13.5 ml test tubes. Each was centrifuged for approximately 12 seconds at 5 g and the supernatant was discarded. Approximately 4.5 ml of EGTA solution was used to re-suspend the pellet in each tube. The contents of both were combined to form the stock suspension.

An initial count was performed on the stock suspension to ensure that the cell count was sufficiently high. In our experience 25×10⁴ cells per ml of suspension was found to be the lowest initial count which would yield distinguishable results after permeabilisation, so the concentration of the stock suspension was altered until it lay above this.

All cell counting described in this appendix was performed using a haemocytometer.

### Ca²⁺-Contraction Assay

Physiologically, myofilaments are activated by a rapid increase in the cytosolic Ca²⁺ concentration from 0.1 μM to approximately 2 μM which occurs mainly due to Ca²⁺ release from the SR (Levick, 2000). The Ca²⁺-contraction assay used in this investigation relies upon the fact that cells with permeabilised membranes allow free access of Ca²⁺ to the cytosol when exposed to high extracellular Ca²⁺ concentrations. This activates the myofilaments of the cell causing it to contract into a ball. Non-permeabilised myocytes retain their characteristic rod shape. The effects of permeabilising agents on the cell population were quantified by counting the proportion of rod-shaped cells.

### Trypan Blue Assay

Access of the vital dye Trypan blue (MW: 950 approx.) into myocytes was used to evaluate permeabilisation by Saponin (Calbiochem brand) or β-escin. Permeabilised myocytes were identified by positive staining (blue colour) of the cell contents. Cells with intact membranes excluded the dye and it was this population that was counted.

### Preparation of Permeabilising Agent Solutions

On each experimental run only one permeabilising agent was investigated. Since both saponin and β-escin degrade in solution within approximately three to four hours it was necessary to make up fresh solutions for each run. Saponin (Calbiochem®, USA; lot no:B31340) or β-Escin (Sigma Chemical Co., St. Louis, USA; lot no:109H0964) were made-up in three stock concentrations, 100 mg/ml, 10 mg/ml and 10 μg/ml (0.01 mg/ml) in distilled water. The quantities of each stock added to obtain the required concentrations when added to 1 ml of cell suspension are shown in Table 2.

### Experimental Protocol

### Ca²⁺-Contraction Assay

On each run utilising the Ca²⁺-contraction assay, sixteen 13.5 ml test tubes were obtained. Eight of these were labelled “EGTA” whilst the remaining eight were labelled “CaEGTA”. Within both sets of eight, individual tubes were labelled control 1, control 2, 0.01, 0.1, 1, 10, 100 and 1000.

The cell stock suspension was agitated to ensure uniform distribution of cells and exactly 1 ml was pipetted into each tube labelled “EGTA”. These tubes were then transferred to the fridge to prevent degradation of the cells.

Each tube was put through the following protocol in turn. The required volume of permeabilising agent was added (see Table 2) and a stopclock was started. During the two minute incubation time, exactly 0.5 mls of the contents of the “EGTA” tube was withdrawn using a pipette and transferred to the corresponding “CaEGTA” tube.

Once the incubation time had elapsed the tubes were centrifuged for approximately 12 seconds at 5 g and the supernatant was withdrawn. The “EGTA” pellet was re-suspended in 0.5 mls of EGTA solution and the “CaEGTA” pellet in 0.5 mls of CeEGTA solution. Both tubes were agitated and the cell density determined by haemocytometer. The protocol was followed for all concentrations under investigation; for the two controls no permeabilising agent was added and thus no incubation step was required.

### Trypan Blue Assay

For each run utilising the trypan blue assay, eight 13.5 ml test tubes were obtained. Each was labelled control 1, control 2, 0.1, 1, 10, 100, 1000 or 10000.

The cell stock suspension was agitated to ensure uniform distribution of cells and exactly 1 ml was pipetted into each tube. These were then transferred to the fridge to prevent degradation of the cells. Each tube was put through the following protocol in turn. The required volume of permeabilising agent was added (see Table 2) and a stopclock was started.

Once the incubation time had elapsed the tube was centrifuged for approximately 12 seconds at 5 g and the supernatant was withdrawn. The pellet was re-suspended in 0.5 mls of EGTA solution and 0.5 mls of 0.5% Trypan blue (BDH Laboratory Supplies, Poole, England) in 5 mM EGTA, which had been previously gently heated to ensure that the dye was dissolved. The tube was agitated and cell density determined by hamocytometer counting.

The protocol was followed for all concentrations under investigation; for the two controls no permeabilising agent was added and thus no incubation step was required.

### Exposure to High Concentrations

The effect of exposing myocytes to exceedingly high concentrations of permeabilising agent (10000 μg/10 mg) was investigated for both β-Escin and saponin. The protocol followed was the same as the Ca²⁺-contraction assay protocol (described above). 100 μl of the 100 mg/ml stock was added to the 1 ml suspension volume to achieve the required concentration (see Table 2). A control plus a low concentration of agent (1 μg/ml) were also studied to validate results.

### Comparison of Two Brands of Saponin

To demonstrate the variation in effect of saponins from different manufacturers, two saponins were investigated; one from Calbiochem (Calbiochem®, USA; lot no:B31340), as used previously, and one from Sigma (Sigma Chemical Co., St. Louis, USA; lot no:19H7841). The concentrations investigated were 1, 10, 100 and 1000 μg/ml for each saponin plus two controls. The experimental protocol followed was the same as for the Ca²⁺-contraction assay method above.

### Confocal Imaging of Cell Permeabilisation

In order to demonstrate the process of permeabilisation, images were taken using laser-scanning confocal microscopy and fluo-3 (Molecular Probes). Fluo-3 is a calcium sensitive fluorescent indicator; local Ca²⁺ concentration is directly proportional to fluorescence intensity. The time-course of permeabilisation was measured by the entry of fluo-3 into the cell. Excitation of fluo-3 occurred at 488 nm whilst emission was measured at 518 nm. Cells were viewed with a 60× water objective lens and Sigma saponin added to give a final concentration of 100 μg/ml. Images were taken every two seconds. The time from addition of permeabilising agent to fully permeabilised state was noted.

### Statistical Analysis of Results

All experimental results were expressed as the mean±s.e. mean of n experiments. These were normalised against either (i) control counts or (ii) initial counts.

Graphs were plotted and non-linear logistic curves were fitted using least squares technique by the Microcal™ Origin™ package (version 5.0, Microcal Software Inc.). ED₅₀ values and their associated errors were also estimated using this software. Analysis of variance (ANOVA) using Tukey's post-test comparison was used to verify significance between ED₅₀ values (GraphPad InStat™, GraphPad Software, V2.05a).

### Results

### Ca²⁺-Contraction Assay

A decrease in rod count in 5 mM Ca²⁺ EGTA indicated that the sarcolemma was hyperpermeable to the high calcium concentration of the bathing medium; this is due to activation of the myofilaments by the high intracellular Ca²⁺ concentration and the development of a hyper-contraction.

In general, exposure of isolated myocytes to the saponins or β-escin caused the rod count to decrease in a concentration-dependent manner.

### Calbiochem Saponin

The results show that Calbiochem saponin produced permeabilisation over the concentration range 10-10000 μg/ml for the Ca²⁺-contraction assay method (FIG. 3). This was evidenced by the large difference between the rod counts in 5 mM EGTA and 5 mM Ca²⁺ EGTA. Between the concentrations 0.01-10 μg/ml, mean counts in 5 mM EGTA and 5 mM Ca²⁺ EGTA were similar; error bars overlapped and therefore means were not considered significantly different (n=4-8). A significant decrease in the rod count in 5 mM Ca²⁺ EGTA was apparent at 10 μg/ml and, over a ten-fold increase in concentration from 10-100 μg/ml, the rod count dropped by approximately 80%. The calculated ED₅₀ value for Calbiochem saponin using this assay system was 45.4±16.2 μg/ml. Approximately 100% of the initial cell population were hyperpermeable at 1000 (n=8) and 10000 μg/ml (n=3). The minimum effective concentration for Calbiochem saponin was 100 μg/ml. A slight trend to increase was noted in the counts in 5 mM EGTA with increasing saponin concentrations.

### Sigma Saponin

The permeabilising effects of Sigma saponin were studied over the concentration range 1-1000 μg/ml; results are presented in FIG. 4. From experience, this was the range over which the permeabilising effect of saponin occurred. Sigma saponin began to hyper-contract, hence permeabilise, the myocytes between 1-10 μg/ml (ED₅₀=5.9±0.4 μg/ml). Approximately 100% of the cells were permeabilised at 100 and 1000 μg/ml. The minimum effective concentration of Sigma saponin was 100 μg/ml. Counts in 5 mM EGTA over the range were stable at approximately 100% (1.0) thus consistent with the control count. Comparison with the Calbiochem brand showed a significant difference (p<0.05).

β-escin

Rod counts were stable in both 5 mM EGTA and 5 mM Ca²⁺ EGTA over the concentration range 0.01-1 μg/ml (n=7-10); these are presented in FIG. 5. The first concentration of β-escin which began to cause permeabilisation was 10 μg/ml (n=6), this was the steepest section of the graph and error bars were greatest here. Permeabilisation was seen between 1-10000 μg/ml; ED₅₀=8.3±1.1 μg/ml. When compared to the ED₅₀ for Sigma saponin no significant difference occurred (p>0.05); in contrast a significant difference was shown when compared to Calbiochem saponin (p<0.05). Approximately 100% of the control population were hyperpermeable to Ca²⁺ at 100, 1000 and 10000 μg/ml β-escin (n=3-7). The minimum effective concentration of β-escin was 100 μg/ml.

Rod counts in 5 mM EGTA were maintained at the control level up to 100 μg/ml (n=7); thereafter they declined over two concentration increments to zero; a Ca²⁺-independent contraction had occurred. The ED₅₀ value for this effect was 1192.1±112.3 μg/ml.

### Trypan Blue Assay

The access of trypan blue into the cell was assessed by plotting the mean percentage of rods in 5 mM EGTA excluding dye at each concentration studied (0.1-10000 μg/ml; n=3). Rods with intact membranes excluded the dye and thus a reduction in the count reflected an increased proportion of rods with hyperpermeable membranes. In general, the number of cells allowing trypan blue access increased as concentration of saponin/β-escin increased.

### Calbiochem Saponin

Between 0.1-10 μg/ml the mean count was stable at approximately 90% (0.9) (FIG. 6). Thereafter the mean count began to decrease. At 1000 and 10000 μg/ml approximately 100% of the control population were permeable to trypan blue. The ED₅₀ for this process was calculated as 46.4±0.9 μg/ml and was not significantly different to the ED₅₀ value for Calbiochem saponin using the Ca²⁺-contraction assay.

β-escin

At 0.1 μg/ml mean rod count was approximately 90% (0.9); this increased at 1 μg/ml to approximately 110% (1.1) (FIG. 7). However, error bars for these two points overlap and so means are not considered to be different. Trypan blue began to enter the myocytes from 1-10 μg/ml; at 10 μg/ml the mean rod count had decreased by approximately 35% of the control value. Approximately 100% of the control population were permeable to dye at 100, 1000 and 10000 μg/ml thus the minimum effective concentration of β-escin was 100 μg/ml. The ED₅₀ for access of trypan blue was calculated as 13.9±2.2 μg/ml β-escin; this was not significantly different from the ED₅₀ value for β-escin using the Ca²⁺-contraction assay.

### Reduction in Cell Numbers Independent of Permeabilising Agent

The cell quality and integrity of their membranes was thought to be a major variable in the study. In addition it was recognised that the protocol itself may have disrupted some of the membranes. To account for these factors the mean count for each concentration was normalised against the initial count. The range 0.01-1 μg/ml was chosen to determine this effect as no decrease due to permeabilisation was demonstrated over this range in the results normalised against the control count. The results for Ca²⁺-contraction assay for Calbiochem saponin, Sigma saponin and β-escin are shown in FIG. 8. The graphs show that for the three agents studied an approximate 10-20% reduction in cell count occurred in 5 mM EGTA whilst this increased to approximately 50-65% in 5 mM Ca²⁺ EGTA.

### Assessment of ED₅₀ Values

ED₅₀ values were estimated from the non-linear logistic curves (FIG. 9) using the standard logistic equation equation. The small Chi-square values obtained (0.00003-0.012) indicate that the fit was reasonable in all cases.

The ED₅₀ is the concentration of agent which permeabilises 50% of the control population. The ED₅₀ values for each agent and associated assay method are shown in Table 3.

### REFERENCES

Afzal, N. & Dhalla, N. S. (1992). Differential changes in left ventricular and right ventricular SR calcium transport in congestive heart failure. American Journal of 25 Physiology 262, H868-H874.
Anversa, P., Olivetti, G., & Capasso, J. M. (1991). Cellular basis of ventricular remodeling after myocardial infarction. American Journal of Cardiology 68, 7D-16D.
Arai, M., Alpert, N. R., MacLennan, D. H., Barton, P., & Periasamy, M. (1993). Alterations in sarcoplasmic reticulum gene expression in human heart failure. Circulation Research 72, 463-469.
Bartel, S., Stein, B., Eschenhagen, T., Mende, U., Neumann, Schmitz, W., Krause, E.-G., Karczewski, P., & Scholz, H. (1996). Protein phosphorylation in isolated trabeculae from nonfailing and failing human hearts. Molecular and Cellular Biochemistry 157, 171-179. 10
Bers, D M (2001) Excitation coupling and cardiac contratile force. 2^{nd} Edition Kluwer Academic Press
Beuckelmann, D. J., Nabauer, M., & Erdmann, E. (1992). Intracellular calcium handling in isolated ventricular myocytes from patients with terminal heart failure. Circulation 85, 1046-1055.
Bing, O. H. L., Brooks, W. W., Conrad, C. H., Sen, S., Perreault, C. L., & Morgan, J. P. (1991). Intracellular calcium transients in myocardium from spontaneously hypertensive rats during the transition to heart failure. Circulation Research 68, 1390-1400.
Brillantes, A.-M., Allen, Takahashi, T., Izumo, S., & Marks, A. R. (1992). Differences in cardiac calcium release channel (ryanodine receptor) expression in myocardium from patients with end-stage heart failure caused by ischemic versus dilated cardiomyopathy. Circulation Research 71, 18-26.
Chapman, R. A. & Tunstall, J. (1983). The control of the strength of the caffeine contracture in frog atrial trabeculae: An activity of the sodium-calcium exchange. Quarterly Journal of Experimental Physiology 68, 381-395.
Coll, K. E. Johnson, R. G. & McKenna, E. (1999) Relationship between phospholamban and nucleotide activation of cardiac sarcoplasmic reticulum Ca²⁺ adenosinetriphosphate. Biochemistry, 38, 2444-2451.
Cory, C. R., McCutcheon, L. J., O'Grady, M., Pang, A. W., Geiger, J. D., & O'Brien, P. J. (1993). Compensatory downregulation of myocardial Ca channel in SR from dogs with heart failure. American Journal of Physiology 264, H926-H937.
Currie, S. & Smith, G. L. (1999). Enhanced phosphorylation of phospholamban and down regulation of SERCA 2 in cardiac sarcoplasmic reticulum from rabbits with heart failure. Cardiovascular Research 41, 135-146.
Gomez, A. M., Valdivia, H. H., Cheng, H., Lederer, M. R., Santana, L. F., Cannell, M. B., McCune, S. A., Altschuld, R. A., & Lederer, W. J. (1997). Defective excitation-contraction coupling in experimental cardiac hypertrophy and heart failure. Science 276, 800-806.
Gwathmey, J. K., Copelas, L., MacKinnon, R., Schoen, F. J., Feldman, M. D., Grossman, W., & Morgan, J. P. (1987). Abnormal intracellular calcium handling in myocardium from patients with end-stage heart failure. Circulation Research 61, 70-76.
Hasenfuss, G. (1998). Animal models of human cardiovascular disease, heart failure and hypertrophy. Cardiovascular Research 39, 60-76.
Hasenfuss, G., Reinecke, H., Studer, R., Meyer, M., Pieske, B., Holtz, J., Holubarsch, C, Posival, H., Just, H., & Drexler, H. (1994). Relation between myocardial function and expression of sacroplasmic reticulum Ca²⁺-ATPase in failing and nonfailing human myocardium. Circulation Research 75, 434-442.
Kiss, E., Ball, N. E., & Kranias, E. G. (1995). Differential changes in cardiac phospholamban and sarcoplasmic reticular Ca-ATPase protein levels—effects on Ca²⁺ transport and mechanics in compensated pressure overload and mechanics in compensated pressure overloaded hypertrophy and congestive heart failure. Circulation Research 11, 759-764.
Marx, S. O., Reiken, S., Hisamatsu, Y., Jayaraman, T., Burkhoff, D., Rosemblit, N., & Marks, A. R. (2000). PKA Phosphorylation Dissociates FKB12.6 from the Calcium Release Channel (Ryanodine Receptor): Defective Regulation in Failing Hearts. Cell 101, 365-376.
Matsui, H., McLennan, D. H., Alpert, N. R., & Periasamy, M. (1995). Sarcoplasmic reticulum gene expression in pressure overlosd-induced cardiac hypertrophy in rabbit. American Journal of Physiology 268, C252-C258.
McDonagh, T. A., Morrison, C. E., Lawrence, A., Ford, I., Tunstallpedoe, H., McMurray, J. J. V., & Dargie, H. J. (1997). Symptomatic and asymptomatic left-ventricular systolic dysfunction in an urban population. Lancet 350, 829-833.
Mercadier, J.-J., Lompre, A.-M., Due, P., Boheler, K. R., Fraysse, J.-B., Wisnewsky, C, Allen, P. D., Komajda, M., & Schwartz, K. (1990). Altered sarcoplasmic reticulum Ca²⁺-ATPase gene expression in the human ventricle during end-stage heart failure. Journal of Clinical Investigation 85, 305-309.
Meyer, M., Schillinger, W., Pieske, B., Holubarsch, C, Heilmann, C, Posival, H., Kuwajima, G., Mikoshiba, K., Just, H., & Hasenfuss, G. (1995). Alterations of SR proteins in failing human dilated cardiomyopathy. Circulation 92, 778-784.
Movsesian, M. A., Bristow, M. R., & Krall, J. (1989). Ca²⁺ uptake by cardiac SR from patients with idiopathic cardiomyopathy. Circulation Research 65, 1141-1144.
Movsesian, M. A., Karimi, M., Green, K., & Jones, L. R. (1994). Ca²⁺ transporting ATPase, phospholamban on dynamics of cardiac myocyte contraction and intracellular Ca²⁺. Circulation 90, 653-657.
Ono, K., Yano, M., Ohkusa, T., Kohno, M., Hisaoka, T., Tanigawa, T., Kobayashi, S., & Matsuzaki, M. (2000). Altered interactions of FKBP12.6 with ryanodine receptor as a cause of abnormal Ca²⁺ release in heart failure. Cardiovascular Research 48, 323-331.
Pieske, B., Kretschmann, B., Meyer, M., Holubarsch, C, Weirich, J., Posival, H., Minami, K., Just, H., & Hasenfuss, G. (1995). Alterations in intracellular calcium handling associated with the inverse force-frequency relation in human dilated cardiomyopathy. Circulation 92, 1169-1178.
Qin, D., Zhang, Z., Caref, E. B., Boutjdir, M., Jain, P., & El-Sherif, N. (1996), Cellular and ionic basis of arrhythmias in postinfarction remodeled ventricular myocardium. Circulation Research 79, 461-473.
Schwinger, R. H. G., Bohm, M., Schmidt, U., Karczewski, P., Bavendiek, U., Flesch, M., Krause, E.-G., & Erdmann, E. (1995). Unchanged protein levels of SERCAII and phospholamban but reduced Ca²⁺ uptake and Ca²⁺-ATPase activity of cardiac SR from dilated cardiomyopathy patients compared with patients with non-failing hearts. Circulation 92, 3220-3228.

1. A method for preparing a stock of cells for future use in an assay for determining activity or function of a target intracellular component, the method comprising contacting a population of cells with an effective amount of a permeabilising agent and freezing the cells.

2. A method according to claim 1 wherein there is substantially no cryoprotectant present in the cell preparation.

3. A method according to claim 2 wherein cryoprotectant is glycerol or DMSO.

4. A method according to claim 1 wherein the cells are myocytes.

5. A method according to claim 4 wherein the cells are cardiac myocytes.

6. A method according to claim 1 wherein the permeabilising agent is selective for the plasma membrane over the intracellular organelle membranes.

7. A method according to claim 6 wherein the permeabilising agent is a saponin.

8. A method according to claim 7 wherein the saponin is beta-escin.

9. A method according to claim 1 wherein the permeabilising agent may be rendered substantially ineffective by a ten-fold dilution of the thawed cell preparation before performing the assay.

10. A method according to claim 1 comprising storing the cells at about −70° C. or below.

11. A method according to claim 1 wherein the cell stock further comprises one or more of: i) an organic buffer; ii) a source of monovalent metal cations, preferably potassium ions.

12. A method according to claim 1 wherein the cell stock further comprises one or more of:
i) an organic buffer; ii) a source of monovalent metal cations, preferably potassium ions; iii) a calcium-specific chelating agent; iv) ATP; v) creatine phosphate; vi) a calcium precipitating agent; vii) a protein kinase inhibitor.

13. A frozen cell preparation, preparable by the method of claim 1.

14. A method for determining an activity of a target intracellular component of a permeabilised cell, the method comprising providing a frozen cell preparation as prepared by the method of claim 1, thawing said cell preparation, and performing an assay for the activity of the target component.

15. A method according to claim 14 wherein the assay comprises the step of contacting the thawed, permeabilised cells with a test substance, and determining the effect of said substance on the activity of the target component.

16. A method according to claim 14 wherein the assay for the activity of the target component is performed directly on the cell preparation.

17. A method according to claim 14 wherein the target component is associated with an intracellular structure or organelle.

18. A method according to claim 17 wherein the target component is associated with myofibrils, nuclei, mitochondria, or sarcoplasmic reticulum.

19. A method according to claim 14 wherein the change in activity is determined by detecting changes in Ca²⁺ levels following contact between the permeabilised cells and the substance under test.

20. A method according to claim 19 wherein the target component is the Ca²⁺ ATPase (SERCA), phospholamban (PIb), ryanodine receptor (RyR), FKBP12.6, Sorcin, Calmodulin, Ca-calmodulin-activated kinase, or cAMP-activated kinase.

21. A method according to claim 14 further comprising the step of contacting the cell preparation with a diluent to form an assay mixture.

22. A method according to claim 21 wherein the method comprises addition of nine volumes of diluent per volume of cell preparation.

23. A method according to claim 21 wherein the diluent does not contain permeabilising agent.

24. A method according to claim 21 wherein the assay for the activity of the target component is performed directly on the assay mixture.

25. A method according to claim. 21 further comprising the step of adding an initiating agent to the assay mixture to begin the assay, wherein the initiating agent is a substance which is required for activity of the target component.

26. A method according to claim 25 wherein the assay is dependent on SERCA activity and the initiating agent comprises magnesium ions.

27. A method according to claim 26 wherein the assay monitors Ca²⁺ release from the SR and the diluent comprises a Ca²⁺-ATPase inhibitor.

28. A method according to claim 21 wherein the assay monitors calcium uptake by the SR and the diluent comprises an inhibitor of calcium release from the SR.

29. A method according to claim 21 wherein the assay mixture comprises an indicator for the presence of calcium ions.

30. A method according to claim 29 wherein the indicator is a fluorescent. dye whose fluorescence changes on contact with calcium.

31. A kit comprising a frozen cell preparation prepared by a method according to claim 1 and a diluent.

32. A kit according to claim 31 wherein the frozen cell preparation comprises: g) cardiac myocytes; h) a source of monovalent metal cations, preferably potassium ions; i) ATP; j) Creatine Phosphate; k) organic buffer, e.g. HEPES;
1) EGTA; g) low affinity Ca²⁺ precipitating agent, e.g. oxalate; h) permeabilising agent, e.g. a saponin.

33. A kit according to claim 32 wherein the diluent comprises:
a) a source of monovalent metal cations, preferably potassium ions, as found in the cell preparation; b) ATP; c) Creatine Phosphate; d) organic buffer as found in the cell preparation; e) EGTA; f) low affinity Ca²⁺ precipitating agent as found in the cell preparation, e.g. oxalate; g) protein kinase inhibitor, e.g. H89.

34. A kit according to claim 32 further comprising a source of magnesium ions.

35. A kit according to claim 32 further comprising an agent for inhibiting release of calcium from the SR.

36. A kit according to claim 31 wherein the diluent comprises:
a) a source of monovalent metal cations, preferably potassium ions, as found in the cell preparation; b) ATP; c) Creatine Phosphate; d) organic buffer as found in the cell preparation; e) EGTA; f) low affinity Ca²⁺ precipitating agent as found in the cell preparation, e.g. oxalate; g) MgCl₂; h) SERCA inhibitor, e.g. thapsigargin; i) protein kinase inhibitor, e.g. H89.

37. A kit according to claim 31 further comprising an agent which provides a detectable signal on contact with calcium ions.
